SEQUENCE LISTING (57 sequences)

SEQ ID NO: 1 - protein, 754 aa, Artificial Sequence
  Description of Artificial Sequence = Synthetic Construct
MWKLLPAAGPAGGEPYRLLTGVEYVVGRKNCAILIENDQSISRNHAVLTANFSVTNLSQTDEIPVLTLKDNSKYGTFVNEEKMQNGFSRTLKSGDGITFGVFGSKFRIEYEPLVACSSCLDVSGKTALNQAILQLGGFTVNNWTEECTHLVMVSVKVTIKTICALICGRPIVKPEYFTEFLKAVESKKQPPQIESFYPPLDEPSIGSKNVDLSGRQERKQIFKGKTFIFLNAKQHKKLSSAVVFGGGEARLITEENEEEHNFFLAPGTCVVDTGITNSQTLIPDCQKKWIQSIMDMLQRQGLRPIPEAEIGLAVIFMTTKNYCDPQGHPSTGLKTTTPGPSLSQGVSVDEKLMPSAPVNTTTYVADTESEQADTWDLSERPKEIKVSKMEQKFRMLSQDAPTVKESCKTSSNNNSMVSNTLAKMRIPNYQLSPTKLPSINKSKDRASQQQQTNSIRNYFQPSTKKRERDEENQEMSSCKSARIETSCSLLEQTQPATPSLWKNKEQHLSENEPVDTNSDNNLFTDTDLKSIVKNSASKSHAAEKLRSNKKREMDDVAIEDEVLEQLFKDTKPELEIDVKVQKQEEDVNVRKRPRMDIETNDTFSDEAVPESSKISQENEIGKKRELKEDSLWSAKEISNNDKLQDDSEMLPKKLLLTEFRSLVIKNSTSRNPSGINDDYGQLKNFKKFKKVTYPGAGKLPHIIGGSDLIAHHARKNTELEEWLRQEMEVQNQHAKEESLADDLFRYNPYLKRRR

SEQ ID NO: 2 - DNA, 4406 bp, Artificial Sequence
  Description of Artificial Sequence = Synthetic Construct
ttcggcacgaggcgcggttgcacgtcggccccagccctgaggagccggaccgatgtggaaactgctgcccgccgcgggcccggcaggaggagaaccatacagacttttgactggcgttgagtacgttgttggaaggaaaaactgtgccattctaattgaaaatgatcagtcgatcagccgaaatcatgctgtgttaactgctaacttttctgtaaccaacctgagtcaaacagatgaaatccctgtattgacattaaaagataattctaagtatggtacctttgttaatgaggaaaaaatgcagaatggcttttcccgaactttgaagtcgggggatggtattacttttggagtgtttggaagtaaattcagaatagagtatgagcctttggttgcatgctcttcttgtttagatgtctctgggaaaactgctttaaatcaagctatattgcaacttggaggatttactgtaaacaattggacagaagaatgcactcaccttgtcatggtatcagtgaaagttaccattaaaacaatatgtgcactcatttgtggacgtccaattgtaaagccagaatattttactgaattcctgaaagcagttcagtccaagaagcagcctccacaaattgaaagtttttacccacctcttgatgaaccatctattggaagtaaaaatgttgatctgtcaggacggcaggaaagaaaacaaatcttcaaagggaaaacatttatatttttgaatgccaaacagcataagaaattgagttccgcagttgtctttggaggtggggaagctaggttgataacagaagagaatgaagaagaacataatttctttttggctccgggaacgtgtgttgttgatacaggaataacaaactcacagaccttaattcctgactgtcagaagaaatggattcagtcaataatggatatgctccaaaggcaaggtcttagacctattcctgaagcagaaattggattggcggtgattttcatgactacaaagaattactgtgatcctcagggccatcccagtacaggattaaagacaacaactccaggaccaagcctttcacaaggcgtgtcagttgatgaaaaactaatgccaagcgccccagtgaacactacaacatacgtagctgacacagaatcagagcaagcagatacatgggatttgagtgaaaggccaaaagaaatcaaagtctccaaaatggaacaaaaattcagaatgctttcacaagacgcacccactgtaaaggagtcctgcaaaacaagctctaataataatagtatggtatcaaatactttggctaagatgagaatcccaaactatcagctttcaccaactaaattgccaagtataaataaaagtaaagatagggcttctcagcagcagcagaccaactccatcagaaactactttcagccgtctaccaaaaaaagggaaagggatgaagaaaatcaagaaatgtcttcatgcaaatcagcaagaatagaaacgtcttgttctcttttagaacaaacacaacctgctacaccctcattgtggaaaaataaggagcagcatctatctgagaatgagcctgtggacacaaactcagacaataacttatttacagatacagatttaaaatctattgtgaaaaattctgccagtaaatctcatgctgcagaaaagctaagatcaaataaaaaaagggaaatggatgatgtggccatagaagatgaagtattggaacagttattcaaggacacaaaaccagagttagaaattgatgtgaaagttcaaaaacaggaggaagatgtcaatgttagaaaaaggccaaggatggatatagaaacaaatgacactttcagtgatgaagcagtaccagaaagtagcaaaatatctcaagaaaatgaaattgggaagaaacgtgaactcaaggaagactcactatggtcagctaaagaaatatctaacaatgacaaacttcaggatgatagtgagatgcttccaaaaaagctgttattgactgaatttagatcactggtgattaaaaactctacttccagaaatccgtctggcataaatgatgattatggtcaactaaaaaatttcaagaaattcaaaaaggtcacatatcctggagcaggaaaacttccacacatcattggaggatcagatctaatagctcatcatgctcgaaagaatacagaactagaagagtggctaaggcaggaaatggaggtacaaaatcaacatgcaaaagaagagtctcttgctgatgatctttttagatacaatccttatttaaaaaggagaagataactgaggattttaaaaagaagccatggaaaaacttcctagtaagcatctacttcaggccaacaaggttatatgaatatatagtgtatagaagcgatttaagttacaatgttttatggcctaaatttattaaataaaatgcacaaaactttgattcttttgtatgtaacaattgtttgttctgttttcaggctttgtcattgcatctttttttcatttttaaatgtgttttgtttattaaatagttaatatagtcacagttcaaaattctaaatgtacgtaaggtaaagactaaagtcacccttccaccattgtcctagctacttggttcccctcagaaaaaaattcatgatactcatttcttatgaatctttccagggatttttgagtcctattcaaattcctatttttaaataatttcctacacaaatgatagcataacatatgcagtgttctacaccttgcttttttacttagtagattaaaaattataggaatatcaatataatgtttttaatattttttcttttccattatgctgtagtcttacctaaactctggtgatccaaacaaaatggcttcagtggtgcagatgtcacctacatgttattctagtactagaaactgaagaccatgtggagacttcatcaaacatgggtttagttttcaccagaatggaaagacctgtacccctttttggtggtcttactgagctgggtgggtgtctgttttgagcttatttagagtcctagttttcctacttataaagtagaaatggtgagattgttttctttttctaccttaaagggagatggtaagaaacaatgaatgtcttttttcaaactttattgacaagtgattttcaagtctgtgttcaaaaatatattcatgtacctgtgatccagcaagaagggagttccagtcaagagtcactacaactgattagttgtttagagaatgagaaatggaacagtgaggaatggaggccatatttccatgacttcccttgtaaacagaagcaacagaagggacaagaggctggcctctacatcactctcaccttccaaatcttgtggaagtgcatctacttgccagaaccaaattaacttacttccaagttctggctgcttgcaggtggaactccagctgcaagggagttagggaaatgaaggtctttttttaaaagcttctcagccttcctagggaacagaaattgggtgagccaatctgcaatttctactacaggcattgagaccagttagattattgaaatattatagagagttatgaacacttaaattatgatagtggtatgacattggatagaacatgggatactttagaagtagaattgacagggcatattagttgatgaaatggagtcatttgagtctcttaatagccatgtatcataattaccaagtgaagctggtggaacatatggtctccattttacagttaaggaatataatggacagattaatattgttctctgtcatgcccacaatccctttctaaggaagactgccctactatagcagtttttatatttgtcaatttatgaatataatgaatgaggagttctggtacctcctgtctttacaaatattgggtgttgtccagtatttttccctttttaaccattccaatcggtgtgtagtgatgtttcattttggttttaatttgtatatccctgatagctataattgggtcatagaaattctttatacattctagatgcaagtctcttgtcggatatatgtattgagatattacacctagtctgtggcttgactgttttctttatgtcttttgatgaatagaagttttaaattttgacaaggtcaaatttatttttttcttttgtttgatattttttctctccaatttaaccccaagatttcagatattctgctctattatataaactttatatttttatatttgtgatctaccttgaattgatatgtatgttgtgaattatggatcagggttctttttttcccccatacaagtatccagtcattgtaacactgtttattgaaagaattatcctttcctcattaaattaccttgccaattagtctcgtgc

SEQ ID NO: 3 - protein, 22 aa, Artificial Sequence
  Description of Artificial Sequence = Synthetic Construct
QHAKEESLADDLFRYNPYLKRR

SEQ ID NO: 4 - protein, 11 aa, Artificial Sequence
  Description of Artificial Sequence = Synthetic Construct
EESLADDLFRY

SEQ ID NO: 5 - protein, 6 aa, Artificial Sequence
  Description of Artificial Sequence = Synthetic Construct
EEDDRY

SEQ ID NO: 6 - protein, 13 aa, Artificial Sequence
  Description of Artificial Sequence = Synthetic Construct
CEEAALDDLCAAE

SEQ ID NO: 7 - protein, 9 aa, Artificial Sequence
  Description of Artificial Sequence = Synthetic Construct
CEEAALDDL

SEQ ID NO: 8 - protein, 15 aa, Artificial Sequence
  Description of Artificial Sequence = Synthetic Construct
VFEEGGDVDDLLDMI

SEQ ID NO: 9 - protein, 11 aa, Artificial Sequence
  Description of Artificial Sequence = Synthetic Construct
VFEEGGDVDDL

SEQ ID NO: 10 - protein, 9 aa, Artificial Sequence
  Description of Artificial Sequence = Synthetic Construct
EEGGDVDDL

SEQ ID NO: 11 - protein, 5 aa, Artificial Sequence
  Description of Artificial Sequence = Synthetic Construct
CNGRC

SEQ ID NO: 12 - protein, 9 aa, Artificial Sequence
  Description of Artificial Sequence = Synthetic Construct
CDCRGDCFC

SEQ ID NO: 13 - protein, 22 aa, Artificial Sequence
  Description of Artificial Sequence = Synthetic Construct
QHAKAASLAAALFAANPYLKRR

SEQ ID NO: 14 - DNA, 66 bp, Artificial Sequence
  Description of Artificial Sequence = Synthetic Construct
ctagagtcgtgcaggacgtgacatctagtgtcgtgcaggcatctagtgtcgtgcaggacgtgcatc

SEQ ID NO: 15 - DNA, 67 bp, Artificial Sequence
  Description of Artificial Sequence = Synthetic Construct
ctaggatgtcacgtcctgcacgacactagatgcctgcacgacactagatgtcacgtcctgcacgact

SEQ ID NO: 16 - DNA, 49 bp, Artificial Sequence
  Description of Artificial Sequence = Synthetic Construct
catggaaggaggaagcagccaaggagaagaccaccaccaccaccaccac

SEQ ID NO: 17 - DNA, 49 bp, Artificial Sequence
  Description of Artificial Sequence = Synthetic Construct
ctaggtggtggtggtggtggtggtcttctccttggctgcttcctccttc

SEQ ID NO: 18 - protein, 9 aa, Artificial Sequence
  Description of Artificial Sequence = Synthetic Construct
RRRRRRRRR

SEQ ID NO: 19 - protein, 16 aa, Artificial Sequence
  Description of Artificial Sequence = Synthetic Construct
RQPKIWFPNRRKPWKK

SEQ ID NO: 20 - protein, 11 aa, Artificial Sequence
  Description of Artificial Sequence = Synthetic Construct
GRKKRRQRPPQ

SEQ ID NO: 21 - protein, 16 aa, Artificial Sequence
  Description of Artificial Sequence = Synthetic Construct
RQIKIWFQNRRMKWKK

SEQ ID NO: 22 - protein, 16 aa, Artificial Sequence
  Description of Artificial Sequence = Synthetic Construct
RQIAIWFQNRRMKWAA

SEQ ID NO: 23 - protein, 9 aa, Artificial Sequence
  Description of Artificial Sequence = Synthetic Construct
RKKRRQRRR

SEQ ID NO: 24 - protein, 21 aa, Artificial Sequence
  Description of Artificial Sequence = Synthetic Construct
TRSSRAGLQFPVGRVHRLLRK

SEQ ID NO: 25 - protein, 26 aa, Artificial Sequence
  Description of Artificial Sequence = Synthetic Construct
GWTLNSAGYLLGKINKALAALAKKIL

SEQ ID NO: 26 - protein, 18 aa, Artificial Sequence
  Description of Artificial Sequence = Synthetic Construct
KLALKLALKALKAALKLA

SEQ ID NO: 27 - protein, 16 aa, Artificial Sequence
  Description of Artificial Sequence = Synthetic Construct
AAVALLPAVLLALLAP

SEQ ID NO: 28 - protein, 10 aa, Artificial Sequence
  Description of Artificial Sequence = Synthetic Construct
VPMLKPMLKE

SEQ ID NO: 29 - protein, 28 aa, Artificial Sequence
  Description of Artificial Sequence = Synthetic Construct
MANLGYWLLALFVTMWTDVGLCKKRPKP

SEQ ID NO: 30 - protein, 18 aa, Artificial Sequence
  Description of Artificial Sequence = Synthetic Construct
LLIILRRRIRKQAHAHSK

SEQ ID NO: 31 - protein, 21 aa, Artificial Sequence
  Description of Artificial Sequence = Synthetic Construct
KETWWETWWTEWSQPKKKRKV

SEQ ID NO: 32 - protein, 18 aa, Artificial Sequence
  Description of Artificial Sequence = Synthetic Construct
RGGRLSYSRRRFSTSTGR

SEQ ID NO: 33 - protein, 15 aa, Artificial Sequence
  Description of Artificial Sequence = Synthetic Construct
SDLWEMMMVSLACQY

SEQ ID NO: 34 - protein, 12 aa, Artificial Sequence
  Description of Artificial Sequence = Synthetic Construct
TSPLNIHNGQKL

SEQ ID NO: 35 - protein, 31 aa, Artificial Sequence
  Description of Artificial Sequence = Synthetic Construct
RRRRRRRRRQHAKEESLADDLFRYNPYLKRR

SEQ ID NO: 36 - protein, 20 aa, Artificial Sequence
  Description of Artificial Sequence = Synthetic Construct
RRRRRRRRREESLADDLFRY

SEQ ID NO: 37 - protein, 15 aa, Artificial Sequence
  Description of Artificial Sequence = Synthetic Construct
RRRRRRRRREEDDRY

SEQ ID NO: 38 - protein, 22 aa, Artificial Sequence
  Description of Artificial Sequence = Synthetic Construct
RRRRRRRRRCEEAALDDLCAAE

SEQ ID NO: 39 - protein, 18 aa, Artificial Sequence
  Description of Artificial Sequence = Synthetic Construct
RRRRRRRRRCEEAALDDL

SEQ ID NO: 40 - protein, 24 aa, Artificial Sequence
  Description of Artificial Sequence = Synthetic Construct
RRRRRRRRRVFEEGGDVDDLLDMI

SEQ ID NO: 41 - protein, 20 aa, Artificial Sequence
  Description of Artificial Sequence = Synthetic Construct
RRRRRRRRRVFEEGGDVDDL

SEQ ID NO: 42 - protein, 18 aa, Artificial Sequence
  Description of Artificial Sequence = Synthetic Construct
RRRRRRRRREEGGDVDDL

SEQ ID NO: 43 - DNA, 72 bp, Artificial Sequence
  Description of Artificial Sequence = Synthetic Construct
caacatgcaaaagaagagtctcttgctgatgatctttttagatacaatccttatttaaaaaggagaagataa

SEQ ID NO: 44 - DNA, 33 bp, Artificial Sequence
  Description of Artificial Sequence = Synthetic Construct
gaagagtctcttgctgatgatctttttagatac

SEQ ID NO: 45 - DNA, 18 bp, Artificial Sequence
  Description of Artificial Sequence = Synthetic Construct
gaagaggatgatagctac

SEQ ID NO: 46 - DNA, 39 bp, Artificial Sequence
  Description of Artificial Sequence = Synthetic Construct
tgtgaagaggcagccctggatgacctctgtgccgcggaa

SEQ ID NO: 47 - DNA, 27 bp, Artificial Sequence
  Description of Artificial Sequence = Synthetic Construct
tgtgaagaggcagccctggatgacctc

SEQ ID NO: 48 - DNA, 45 bp, Artificial Sequence
  Description of Artificial Sequence = Synthetic Construct
gtatttgaagaaggtggtgatgtggacgatttattggacatgata

SEQ ID NO: 49 - DNA, 33 bp, Artificial Sequence
  Description of Artificial Sequence = Synthetic Construct
gtatttgaagaaggtggtgatgtggacgattta

SEQ ID NO: 50 - DNA, 27 bp, Artificial Sequence
  Description of Artificial Sequence = Synthetic Construct
gaagaaggtggtgatgtggacgattta

SEQ ID NO: 51 - protein, 3056 aa, Artificial Sequence [sequence omitted]

SEQ ID NO: 52 - protein, 14 aa, Artificial Sequence
  Description of Artificial Sequence = Synthetic Construct
AKEESLADDLFRYN

SEQ ID NO: 53 - protein, 9 aa, Artificial Sequence
  Description of Artificial Sequence = Synthetic Construct
KEESLADDL

SEQ ID NO: 54 - protein, 9 aa, Artificial Sequence
  Description of Artificial Sequence = Synthetic Construct
SDADLEELK

SEQ ID NO: 55 - protein, 10 aa, Artificial Sequence
  Description of Artificial Sequence = Synthetic Construct
XEEXXXDDLX

SEQ ID NO: 56 - protein, 273 aa, Artificial Sequence
  Description of Artificial Sequence = Synthetic Construct
RVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDRE

SEQ ID NO: 57 - protein, 335 aa, Artificial Sequence
  Description of Artificial Sequence = Synthetic Construct
HRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTS

(19) United States
(12) Patent Application Publication
(10) Pub. No.: US 2010/0120679 A1
(43) Pub. Date: May 13, 2010

(54) Targeting NBS1-ATM Interaction To Sensitize Cancer Cells To Radiotherapy And Chemotherapy

(75) Inventors: Bo Xu, Hoover, AL (US); Michael J. Cariveau, Mount Olive, NC (US)

Correspondence Address: Ballard Spahr LLP, SUITE 1000, 999 PEACHTREE STREET, ATLANTA, GA, 30309-3915, US

(73) Assignee: SOUTHERN RESEARCH INSTITUTE, Birmingham, AL (US)

(21) Appl. No.: 12/447,717

(22) PCT Filed: Oct. 30, 2007
(86) PCT No.: PCT/US07/22886

§ 371 Date: Jul. 1, 2009

Related U.S. Application Data
(60) Provisional application No. 60/863,457, filed on Oct. 30, 2006.

(51) Int. Cl. A61K 38/16; C07K 14/00; C07K 7/06; C07K 7/08; C07H 21/00; C12N 15/63; C12N 5/00; A61K 31/7088; A61K 38/08; A61K 38/10

(52) U.S. Cl. 514/12; 530/350; 530/326; 530/327; 530/329; 530/328; 536/23.1; 435/320.1; 435/325; 514/44 R; 514/13; 514/14; 514/15; 514/17

(57) ABSTRACT

Provided herein are compositions and methods for use in sensitizing cancer cells to radiation and chemotherapy.

### CROSS-REFERENCE TO RELATED APPLICATIONS

This application claims benefit of U.S. Provisional Application No. 60/863,457, filed Oct. 30, 2006, which is hereby incorporated herein by reference in its entirety.

### BACKGROUND

Of the estimated 1.3 million patients with newly diagnosed cancer in the United States, two-thirds of them will eventually receive some form of radiation therapy as part of their treatment regimen. Radiotherapy is considered one of the most important and powerful treatments for many cancers, especially for localized cancers that have no metastases. Among tumors to be treated by radiotherapy, only a few are highly responsive, including the lymphomas and seminomas. However, many other solid tumors, such as melanoma, glioblastoma, and prostate cancer etc., are typically very resistant to radiation, and they tend to progress even after high dose radiation. Treatment regimens often become more complicated when radiation oncologists consider normal tissue damage, a response that limits the dose and number of fractions in a treatment protocol. Reasons for treatment failure are often multiple and varied (Pawlik and Keyomarsi, 2004). Tumor factors, such as location, size, and inadequate vascular supply (hypoxia), can all play a role in the lack of responsiveness of neoplasms to ionizing radiation (IR). Perhaps most important are the cellular and genetic factors that are related to radiosensitivity regulation, such as differential tissue-specific gene expression, that may result in radiation-resistant cellular phenotypes. Scientists have a long history of developing a variety of methods to increase tumor cell sensitivity to IR. These include hypoxic radiosensitizers, high concentrations of oxygen, and more recently, by targeting many of the genetics factors involved in radiosensitivity (Choudhury et al., 2006). However, even with decades of scientific breakthrough in the field of molecular biology and biochemistry, cancer genetics and molecular radiobiology, limited progress has been made in terms of developing efficient and specific radiosensitizers.

### BRIEF SUMMARY

In accordance with the purpose of this invention, as embodied and broadly described herein, this invention relates to novel radiosensitizers and methods of making and use thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows development of the NBS1 inhibitory peptides. FIG. 1A shows schematic illustration of functional domains of ATM and NBS1 and their interaction. The C-terminal of NBS1 is required for ATM activation and recruitment to sites of DNA damage. It consists of at least two sets of amino acid residues, 736-737 (EE) and 741-743 (DDL), that are evolutionarily conserved and necessary for ATM binding. NBS1 binds to two sets of the Heat Repeats (Heat Repeat 2 (a.a. 248-522), and Heat Repeat 7 (a.a. 1436-1770), in ATM. FIG. 1B shows the amino acid sequences for the R₉, wtNIP, and scNIP peptides developed.

FIG. 2 shows peptide internalization and cytotoxicity. FIG. 2A shows HeLa cells were treated with 10 μM R₉, wtNIP, or scNIP for one hour and analyzed by immunofluorescence microscopy after staining with Fluorescein-conjugated streptavidin.

FIG. 2B shows HeLa cells were treated with Taxol and the NIP peptides at indicated doses. 24 hours after treatment, cell survival was quantified by a standard MTT assay.

FIG. 3 shows wtNIP inhibits NBS1-ATM binding. HeLa cells treated with the NIP peptides were irradiated (0 or 6Gy). Immunoprecipitation was performed with a rabbit NBS1 antibody, and Western blotting was performed with monoclonal antibodies against ATM, NBS1 or MRE11.

FIG. 4 shows wtNIP can inhibit γ-H2AX focus formation. FIG. 4A shows HeLa cells were treated with 10 μM R₉, wtNIP, or scNIP for one hour, irradiated with 0 or 6Gy, and harvested 30 minutes later before immunofluorescence microscopy was employed to detect radiation induced-γ-H2AX foci. FIG. 4B shows the mean γ-H2AX nuclear foci per nucleus determined for each image using Image Pro 5.1 software, expressed in arbitrary units. Error bars represent +/−1 SD, graphed are the mean of three independent experiments.

FIG. 5 shows exposure to the wtNIP peptide abrogates IR-induced NBS1 phosphorylation. FIG. 5A shows HeLa cells were treated with 10 μM R₉, wtNIP, or scNIP for one hour, irradiated with 0 or 6Gy, and harvested 120 minutes later before immunofluorescence microscopy was employed to detect radiation induced-NBS1 focus formation using an anti-Ser343 NBS1 antibody. FIG. 4B shows the mean number of NBS1 foci per nucleus determined from a population of at least 25 cells in three independent experiments. Error bars represent +/−1 SD, graphed are the mean of three independent experiments.

FIG. 6 shows wtNIP increases cellular radiosensitivity. FIG. 6A shows cells seeded at limiting dilutions and treated with 10 μM R₉, wtNIP, or scNIP for one hour prior to irradiation, continuously exposed to the peptides for 24 hours, harvested 10-12 days later, and stained with crystal violet. Shown in A (HeLa), C (MO59J) and D (GM9607) are the susvival curves after indicated doses of radiation. Error bars represent +/−1 SEM, graphed are the mean of three independent experiments. FIG. 6B shows representative plates of the clonogenic assay for NIP mediated radiosensitivity in HeLa cells.

FIG. 7 shows degradation of the R₉, wtNIP or scNIP peptides. HeLa cells were treated with 10 μM R₉, wtNIP, or scNIP for one hour and harvested at indicated time points before they were analyzed by immunofluorescence microscopy staining with an anti-streptavidin antibody.

FIG. 8 shows wtNIP inhibits γ-H2AX focus formation in the prostate cancer cell line DU-145. FIG. 8A shows DU-145 cells were treated with 10 μM R₉, wtNIP, or scNIP for one hour, irradiated with 0 or 6Gy, and harvested 30 minutes later before immunofluorescence microscopy was employed to detect radiation induced-γ-H2AX foci.

FIG. 8B shows the mean γ-H2AX nuclear foci per nucleus determined for each image using Image Pro 5.1 software and is expressed in arbitrary units. Error bars represent +/−1 SD, graphed are the mean of three independent experiments.

FIG. 9 shows exposure to the wtNIP peptide abrogates IR-induced NBS1 phosphorylation in the prostate cancer cell line DU-145. FIG. 9A shows DU-145 cells were treated with 10 μM R₉, wtNTP, or scNIP for one hour, irradiated with 0 or 6Gy, and harvested 120 minutes later before immunofluorescence microscopy was employed to detect radiation induced-NBS1 foci formation using an anti-Ser343 NBS1 antibody.

FIG. 9B shows the mean number of NBS1 foci per nucleus was determined from a population of at least 25 cells in three independent experiments. Error bars represent +/−1 SD, graphed are the mean of three independent experiments.

FIG. 10 shows fluorescence polarization with bound and free Texas red labeled NBS1 peptides.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

The disclosed method and compositions may be understood more readily by reference to the following detailed description of particular embodiments and the Examples included therein and to the Figures and their previous and following description.

### A. COMPOSITIONS

1. ATM-Mediated DNA Damage Response

Disclosed herein are compositions and methods for inhibiting the activation of ATM in order to increase the sensitivity of a cell, such as a cancer cell, to radiotherapy and chemotherapy. Radiotherapy also has applications in non-malignant conditions, such as the treatment of trigeminal neuralgia, severe thyroid eye disease, pterygium, prevention of keloid scar growth, and prevention of heterotopic ossification.

In modern molecular radiation oncology, biological targeting requires in-depth understanding of the mechanism of cellular responses pertaining to cell proliferation, DNA repair and cell death. It is well known that cellular responses to irradiation (IR)-induced DNA damage are controlled by a concisely organized signal transduction network. This network is composed of a number of gene products, which include sensors, transducers and effectors. DNA double strand breaks (DSBs) are detected by sensor molecules that trigger the activation of transducing kinases. These transducers then phosphorylate effector molecules to regulate signaling cascades that control cell cycle checkpoints, influence DNA repair machinery, or trigger apoptotic pathways. One central element in the network is the ATM protein, mutation of which contributes to the human autosomal recessive disorder named ataxia-telangiectasia (A-T) (Shiloh, 2003). A-T is characterized by progressive neuro-degeneration, a variable immunodeficiency, an extremely high predisposition to the development of lymphoid malignancies and hypersensitivity to IR. Cells derived from A-T patients show a variety of abnormalities, including cell cycle checkpoint defects, chromosomal instability and hypersensitivity in response to IR. The gene responsible for the disease was cloned in 1995, and named Ataxia Telangiectasia Mutated (ATM) (Savitsky et al., 1995). The ATM gene is remarkable for its large size and the existence of a sequence in its carboxyl terminus similar to PI-3 kinases. A family of genes, including Tell, Mec1 and Rad3 in yeast, Mei-41 in Drosophila and ATR and DNA-PK in vertebrates, are similar in size and carboxyl terminal kinase sequence, and are all involved in controlling DNA damage responses (Abraham, 2001). The ATM gene encodes a 370-KD protein kinase, which consists of several functional domains, including an FAT domain that is conserved among the above mentioned PI3 kinases and may function as a protein-protein interaction domain. The kinase domain can phosphorylate serine/threonine followed by glutamine (the S/T-Q consensus sequence), and the FAT carboxy-terminal domain (FATC) may regulate protein activity and stability. Previous studies demonstrated that after DNA damage an intermolecular autophosphorylation of ATM on Serine 1981, one of the serine sites within the FAT domain, leads to dissociation of the inactive dimer to an active monomer form of ATM (Bakkenist and Kastan, 2003). However, mouse studies have revealed that ATM with a serine to alanine mutation at Ser1987, the conserved serine residue in mouse, is fully functional in terms of restoring ATM-mediated DNA damage responses (Pellegrini et al., 2006). Therefore the role of Ser1981 autophosphorylation in ATM activation has been ruled out.

The other model for ATM activation is based on the fact that ATM activation is impaired in the absence of NBS1 and Mre11, both of which form a complex with Rad50 (the so-called MRN complex). The MRN complex is highly conserved, influencing each aspect of chromosome break metabolism and is considered a DNA damage sensor to detect strand breaks (Difilippantonio et al., 2005; Dupre et al., 2006). A conserved C-terminus motif of NBS1 binds to several HEAT repeats of ATM, an interaction that is essential to activate the kinase (Falck et al., 2005). Studies have shown that the MRN complex can detect DNA double strand breaks and recruit ATM to damaged DNA molecules (Lee and Paull, 2004; Lee and Paull, 2005; Difilippantonio et al., 2005). Activated ATM can phosphorylate a number of downstream targets to facilitate optimal cellular responses. Over the last ten years, many proteins that are essential for optimal cellular response to DNA damage have been recognized as ATM enzymatic substrates. A good example of this continuously growing ATM substrate list is the most recent finding that the E3 ubiquitin ligase COP1 can be phosphorylated by ATM and its subsequent function is to stabilize p53 in response to DNA damage (Dornan et al., 2006).

Because ATM is central to the cellular response to irradiation, blocking its activation or activity can make virtually any type of tumor much more sensitive to radiation. Since cloning the gene in 1995, a number of investigators have employed several methods to target ATM. These methods include antisense RNA, small interfering RNA (siRNA), and screening of small molecule inhibitors of ATM. Zhang et al., successfully sublconed the full length cDNA of ATM in the opposite orientation into CB3AR cells, where it was shown to significantly increase radiosensitivity. The anti-sense construct imparted an approximate 3-fold increase in radiation sensitivity, similar to that observed in A-T cells. There was an increase in the number of chromosome breaks and apparent radiation resistant DNA synthesis in transfected cells (Zhang et al., 1998). In addition, the radiosensitivity conferred by antisense ATM in glioblastoma and prostate adenocarcinoma cells has been shown to be as much as 4 times higher than that observed in untransfected cells (Guha et al., 2000; Fan et al., 2000).

The development of siRNA recently led to the generation of an siRNA that could inhibit ATM function in prostate cancer cells. Collis et al. designed, and delivered, an exogenous plasmid encoding siRNA's targeting ATM in human cancer cells. Both DU-145 and PC-3 cells, when transfected with these plasmids, exhibited an increase in radiosensitivity at clinically relevant radiation doses (Collis et al., 2003). More recently, stable transfection of Hela cells with an ATM specific siRNA led to a 10-fold increase in sensitivity to ionizing radiation. It has also been shown that ATM silencing in p53 deficient cells leads to a compromise in cell cycle checkpoints, and when combined with doxorubicin, chemosensitivity enhancements as much as 3.1 have been observed (Mukhopadhyay et al., 2005). While siRNA has shown promising results in vivo, transition to clinic studies has been slow.

By screening a combinatorial library of compounds around the DNA-PK inhibitor LY294002, Hickson et al. reported a compound (KU55933) to selectively inhibit the ATM kinase. Their studies have shown a significant increase in radiosensitivity in Hela cells, and as much as 35.5 fold increases in sensitivity to etoposide (Hickson et al., 2004). However the in vivo radiosensitization effect and the toxicity of the compounds have not been reported. Several obstacles of applying the above referenced methods exist, including: 1) Genetic manipulation of the ATM gene by the antisense strategy or the siRNA technique is cumbersome in a clinical setting because of the large size of the gene; 2) these methods do not guarantee tumor specific targeting; therefore an increase in the therapeutic index is uncertain; and 3) more importantly, due to the pleitropic effects of the mutation of the gene, the outcome of directly targeting ATM kinase activity can be complicated, as it is unclear whether the only effect of these reagents will be to confer radiosensitization.

2. Small Inhibitory Peptides

Since NBS1-ATM interaction is important for IR-induced activation of ATM and limiting radiosensitivity, disclosed herein is an approach for developing radiosensitizers that selectively disrupt the signaling pathway. One provided method is the use of small non-functional peptides to block NBS1-ATM interaction. For example, a small peptide containing the wild-type C-terminal NBS1 sequence can inhibit NBS1-ATM interaction and ATM activation. Similarly, a small peptide comprising the heat repeat sequences of ATM can inhibit NBS1-ATM interaction and ATM activation.

The terms “peptide” and “polypeptide” are used herein synonymously to refer to a polymer of two or more amino acids and are not meant to denote a particular length or method of making.

Thus, provided herein is an isolated polypeptide comprising a carboxy-terminal amino acid sequence of NBS1, or a conservative variant thereof (also referred to herein as NIP). For example, the provided peptide can comprise amino acids 734 to 754 of SEQ ID NO:1. The provided peptide can comprise a conservative amino acid substitution within the C-terminal-most 4 to 30 amino acids, including amino acids 734 to 754, of NBS1 (SEQ ID NO:1). In this context, the peptide can comprise 1, 2 or 3 conservative amino acid substitutions. In some aspects, the peptide comprises the amino acid sequence xEExxxDDLx, where x is any amino acid (SEQ ID NO:55).

The peptide can comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The polypeptide can comprise an amino acid sequence with at least 95% sequence identity to SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

Also provided herein is an isolated polypeptide comprising the NBS1-binding sequences of ATM, or a conservative variant thereof. For example, the polypeptide can comprise the heat repeat sequences of ATM, or a fragment thereof, that binds NBS1. For example, the provided polypeptide can comprise SEQ ID NO:56. The provided polypeptide can comprise SEQ ID NO:57. The polypeptide can comprise an amino acid sequence with at least 95% sequence identity to SEQ ID NO:56, or a fragment thereof, that binds NBS1. The polypeptide can comprise an amino acid sequence with at least 95% sequence identity to SEQ ID NO:57, or a fragment thereof, that binds NBS1.

As disclosed herein, the provided polypeptides can inhibit the binding of ATM to the carboxy-terminus of NBS1. Also as disclosed herein, the provided polypeptides can increase the sensitivity of cells, such as cancer cells, to radiotherapy and chemotherapy. Thus, in one aspect, the herein provided isolated polypeptides are in a pharmaceutical composition suitable for administration to a subject.

In one aspect, the herein provided polypeptide can be any polypeptide comprising the carboxy-terminal most amino acids of NBS1, provided that the peptide is not the full-length NBS1. Thus, the provided polypeptide can comprise the C-terminal-most 4 to 30 amino acids of NBS1, including the C-terminal most 4, 5, 6, 7, 8, 9, 10, 11, 12, 13, 14, 15, 16, 17, 18, 19, 20, 21, 22, 23, 24, 25, 26, 27, 28, 29, 30 amino acids of NBS1, or a fragment thereof. For example, the provided polypeptide can comprise amino acids 734 to 754 of NBS1 (SEQ ID NO:1). The provided polypeptide can comprise a conservative amino acid substitution within the C-terminal-most 4 to 30 amino acids, including amino acids 734 to 754, of NBS1 (SEQ ID NO:1). In this context, the peptide can comprise 1, 2 or 3 conservative amino acid substitutions. The polypeptide can comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The polypeptide can comprise an amino acid sequence with at least 95% sequence identity to SEQ ID NO:3, SEQ ID NO:4, SEQ ID NO:5, SEQ ID NO:6, SEQ ID NO:7, SEQ ID NO:8, SEQ ID NO:9, or SEQ ID NO:10. The polypeptide can comprise an amino acid sequence with at least 96% sequence identity to SEQ ID NO:3, SEQ ID NO:4, SEQ ID NO:5, SEQ ID NO:6, SEQ ID NO:7, SEQ ID NO:8, SEQ ID NO:9, or SEQ ID NO:10. The polypeptide can comprise an amino acid sequence with at least 97% sequence identity to SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The polypeptide can comprise an amino acid sequence with at least 98% sequence identity to SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The polypeptide can comprise an amino acid sequence with at least 99% sequence identity to SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

In an alternative aspect, the polypeptide does not comprise the C-terminal most 1, 2, 3, 4, 5, 6, 7, 8, 9, or 10 amino acids, for example. Thus, the peptide can comprise amino acids 734-753, 734-752, 734-751, 734-750, 734-749, 734-748, 734-747, 734-746, 734-745, 734-744 of NBS1. Thus, the peptide can in some aspects not comprise amino acids 754, 753, 752, 751, 750, 749, 748, 747, 746, 745 of NBS1.

The C-terminal of NBS1 is required for ATM activation and recruitment to sites of DNA damage. It comprises at least two sets of amino acid residues, 736-737 (EE) and 741-743 (DDL), that are evolutionarily conserved and necessary for ATM binding. Thus, the NBS1 can comprises the amino acid sequence xEExxxDDLx, where x is any amino acid sequence (SEQ ID NO:55).

In a further aspect, the herein provided polypeptide can be any polypeptide comprising the NBS1-binding domain of ATM, provided that the peptide is not the full-length ATM. Thus, the herein provided polypeptide can be any polypeptide comprising heat repeats 2 and/or 7 of ATM. Thus, the herein provided polypeptide can be any polypeptide comprising amino acids 248-522 of the ATM sequence disclosed in SEQ ID NO:51. Thus, the herein provided polypeptide can be any polypeptide comprising amino acids SEQ ID NO:56. Thus, the herein provided polypeptide can be any polypeptide comprising amino acids 1436-1770 of ATM (SEQ ID NO:51). Thus, the herein provided polypeptide can be any polypeptide comprising amino acids SEQ ID NO:57. The provided polypeptide can comprise a conservative amino acid substitution within the heat repeats 2 and/or 7 of ATM. In this context, the peptide can comprise 1, 2 or 3 conservative amino acid substitutions. The polypeptide can comprise an amino acid sequence with at least 95% sequence identity to SEQ ID NO:56 or SEQ ID NO:57. The polypeptide can comprise an amino acid sequence with at least 96% sequence identity to SEQ ID NO:56 or SEQ ID NO:57. The polypeptide can comprise an amino acid sequence with at least 97% sequence identity to SEQ ID NO:56 or SEQ ID NO:57. The polypeptide can comprise an amino acid sequence with at least 98% sequence identity to SEQ ID NO:56 or SEQ ID NO:57. The polypeptide can comprise an amino acid sequence with at least 99% sequence identity to SEQ ID NO:56 or SEQ ID NO:57.

The provided polypeptide can further constitute a fusion protein or otherwise have additional N-terminal, C-terminal, or intermediate amino acid sequences, e.g., linkers or tags. “Linker”, as used herein, is an amino acid sequences or insertion that can be used to connect or separate two distinct polypeptides or polypeptide fragments, wherein the linker does not otherwise contribute to the essential function of the composition. A polypeptide provided herein, can have an amino acid linker comprising, for example, the amino acids GLS, ALS, or LLA. A “tag”, as used herein, refers to a distinct amino acid sequence that can be used to detect or purify the provided polypeptide, wherein the tag does not otherwise contribute to the essential function of the composition. The provided polypeptide can further have deleted N-terminal, C-terminal or intermediate amino acids that do not contribute to the essential activity of the polypeptide.

3. Fusion Proteins

The herein disclosed polypeptide can be a fusion protein. Fusion proteins, also know as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6xhis-tag) which can be isolated using nickel or cobalt resins (affinity chromatography). Chimeric proteins can also be manufactured with toxins or anti-bodies attached to them in order to study disease development.

Alternatively, internal ribosome entry sites (IRES) elements can be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed Using a single promoter/enhancer to transcribe a single message (U.S. Pat. Nos. 5,925,565 and 5,935,819; PCT/US99/05781). IRES sequences are known in the art and include those from encephalomycarditis virus (EMCV) (Ghattas, I. R. et al., Mol. Cell. Biol., 11:5848-5849 (1991); BiP protein (Macejak and Sarnow, Nature, 353:91 (1991)); the Antennapedia gene of drosophilia (exons d and e) [Oh et al., Genes & Development, 6:1643-1653 (1992)); those in polio virus [Pelletier and Sonenberg, Nature, 334:320325 (1988); see also Mountford and Smith, TIG, 11:179-184 (1985)).

4. Internalization Sequences

The NBS1 peptide can be linked to an internalization sequence or a protein transduction domain to effectively enter the cell. Recent studies have identified several cell penetrating peptides, including the TAT transactivation domain of the HIV virus, antennapedia, and transportan that can readily transport molecules and small peptides across the plasma membrane (Schwarze et al., 1999; Derossi et al., 1996; Yuan et al., 2002). More recently, polyarginine has shown an even greater efficiency of transporting peptides and proteins across the plasma, membrane making it an attractive tool for peptide mediated transport (Fuchs and Raines, 2004). Nonaarginine (R₉, SEQ ID NO:18) has been described as one of the most efficient polyarginine based protein transduction domains, with maximal uptake of significantly greater than TAT or antennapeadia. Peptide mediated cytotoxicity has also been shown to be less with polyarginine-based internalization sequences. R₉ mediated membrane transport is facilitated through heparan sulfate proteoglycan binding and endocytic packaging. Once internalized, heparan is degraded by heparanases, releasing R₉ which leaks into the cytoplasm (Deshayes et al., 2005). Studies have recently shown that derivatives of polyarginine can deliver a full length p53 protein to oral cancer cells, suppressing their growth and metastasis, defining polyarginine as a potent cell penetrating peptide (Takenobu et al., 2002).

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 1).

TABLE 1
Cell Internalization Transporters

| Name | Sequence | SEQ ID NO |
|---|---|---|
| Polyarginine | RRRRRRRRR | SEQ ID NO: 18 |
| Antp | RQPKIWFPNRRKPWKK | SEQ ID NO: 19 |
| HIV-Tat | GRKKRRQRPPQ | SEQ ID NO: 20 |
| Penetratin | RQIKIWFQNRRMKWKK | SEQ ID NO: 21 |
| Antp-3A | RQIAIWFQNRRMKWAA | SEQ ID NO: 22 |
| Tat | RKKRRQRRR | SEQ ID NO: 23 |
| Buforin II | TRSSRAGLQFPVGRVHRLLRK | SEQ ID NO: 24 |
| Transportan | GWTLNSAGYLLGKINKALAALA | SEQ ID NO: 25 |
| | KKIL | |
| model amphipathic | KLALKLALKALKAALKLA | SEQ ID NO: 26 |
| peptide (MAP) | | |
| K-FGF | AAVALLPAVLLALLAP | SEQ ID NO: 27 |
| Ku70 | VPMLK- PMLKE | SEQ ID NO: 28 |
| Prion | MANLGYWLLALFVTMWTDVGL | SEQ ID NO: 29 |
| | CKKRPKP | |
| pVEC | LLIILRRRIRKQAHAHSK | SEQ ID NO: 30 |
| Pep-1 | KETWWETWWTEWSQPKKKRKV | SEQ ID NO: 31 |
| SynB1 | RGGRLSYSRRRFSTSTGR | SEQ ID NO: 32 |
| Pep-7 | SDLWEMMMVSLACQY | SEQ ID NO: 33 |
| HN-1 | TSPLNIHNGQKL | SEQ ID NO: 34 |
| BGSC (Bis- Guanidinium- Spermidine- Cholesterol) | 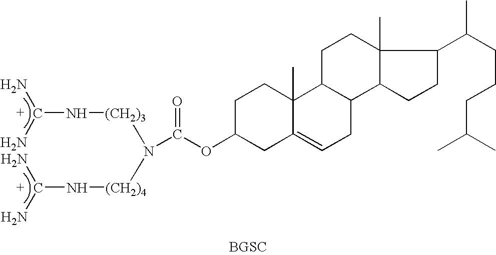 | |
| BGTC (Bis- Guanidinium-Tren- Cholesterol) | 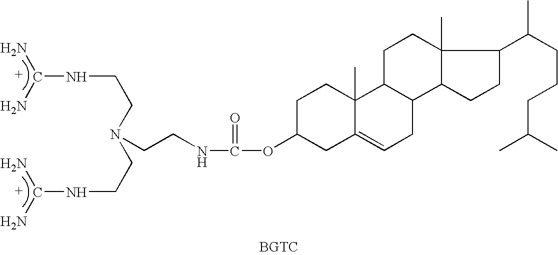 | |

Thus, the provided polypeptide can further comprise the amino acid sequence SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, or SEQ ID NO:34. See Bucci, M. et al. 2000. Nat. Med. 6, 1362-1367; Derossi, D., et al. 1994. Biol. Chem. 269, 10444-10450; Fischer, P. M. et al. 2000. J. Pept. Res. 55, 163-172; Frankel, A. D. & Pabo, C. 0.1988. Cell 55, 1189-1193; Green, M. & Loewenstein, P. M. 1988. Cell 55, 1179-1188; Park, C. B., et al. 2000. Proc. Natl. Acad. Sci. USA 97, 8245-8250; Pooga, M., et al. 1998. FASEB J. 12, 67-77; Oehlke, J. et al. 1998. Biochim. Biophys. Acta. 1414, 127-139; Lin, Y. Z., et al. 1995. J. Biol. Chem. 270, 14255-14258; Sawada, M., et al. 2003. Nature Cell Biol. 5, 352-357; Lundberg, P. et al. 2002. Biochem. Biophys. Res. Commun. 299, 85-90; Elmquist, A., et al. 2001. Exp. Cell Res. 269, 237-244; Morris, M. C., et al. 2001. Nature Biotechnol. 19, 1173-1176; Rousselle, C. et al. 2000. Mol. Pharmacol. 57, 679-686; Gao, C. et al. 2002. Bioorg. Med. Chem. 10, 4057-4065; Hong, F. D. & Clayman, G. L. 2000. Cancer Res. 60, 6551-6556). The provided polypeptide can further comprise BGSC (Bis-Guanidinium-Spermidine-Cholesterol) or BGTC (Bis-Guanidinium-Tren-Cholesterol) (Vigneron, J. P. et al. 1998. Proc. Natl. Acad. Sci. USA. 93, 9682-9686). The preceding references are hereby incorporated herein by reference in their entirety for the teachings of cellular internalization vectors and sequences. Any other internalization sequences now known or later identified can be combined with a peptide of the invention.

For example, the polypeptide can comprise the carboxy-terminal most amino acids of NBS1 and a polyarginine internalization sequence. Thus, for example, the polypeptide can comprise the amino acid sequence SEQ ID NO:35, SEQ ID NO:36, SEQ ID NO:37, SEQ ID NO:38, SEQ ID NO:39, SEQ ID NO:40, SEQ ID NO:41, or SEQ ID NO:42.

5. Tumor-Specific Targeting

A preferred radiosensitizer only sensitizes tumor cells, and spares normal cells. One approach to achieve this is to utilize polypeptides (e.g., modified or fusion proteins) that have both internalization and tumor specific targeting abilities. Since tumor vasculature differs from the vasculature of surrounding normal tissues, both in morphology and biochemistry, this difference has received increased attention in recent years as an important determinant of tumor progression and as a potential target for novel anti-cancer therapies. Biochemically, tumor blood vessels distinguish themselves from resting vessels by expressing a number of angiogenesis-related molecules such as certain integrins, endothelial cell growth factor receptors, proteases, cell surface proteoglycans and extracellular matrix components (Ruoslahti, 2000). In vivo screening of phage display libraries for peptides that home to tumor vasculature when injected into mice has suggested several motifs for tumor homing. They include RGD in the cyclic peptides CDCRGDCFC (SEQ ID NO:12), and NGR the cyclic tumor-homing peptide, CNGRC (SEQ ID NO:11). The NGR-containing peptides have proven useful for delivering cytotoxic drugs, pro-apoptotic peptides, and the tumor necrosis factor α to tumor vasculature (Ellerby et al., 1999; Arap et al., 1998; Arap et al., 2002; Curnis et al., 2002). A third motif, GSL, was also isolated frequently in screens with various types of tumors (Arap et al., 1998). The tumor homing of the phage carrying the RGD, NGR and GSL motif peptides is independent of the tumor type (Arap et al., 1998; Pasqualini et al., 2000; Pasqualini et al., 1997), rather, the homing depends on the angiogenic characteristics of the tumor vasculature. The receptor for the NGR tumor homing peptides is not an integrin. Instead, aminopeptidase N (APN or CD13) has been identified as the receptor for the NGR motif peptides in tumor vasculature (Pasqualini et al., 2000). The NGR-containing peptides have proven useful for delivering cytotoxic drugs, pro-apoptotic peptides, and the tumor necrosis factor α to tumor vasculature (Ellerby et al., 1999; Arap et al., 1998; Arap et al., 2002; Curnis et al., 2002). More interestingly, it has been shown that NGR peptides can bind to prostatic primary and metastatic tumors, but not to normal prostate tissues (Pasqualini et al., 2000). The NGR peptide also displays the ability for cytosolic internalization (Arap et al., 1998).

Any molecule that can target a specific tissue can be used as the targeting molecule of the present polypeptide (e.g., in a fusion protein comprising an NIP). For example, the targeting molecule can be a molecule (e.g., an antibody or aptamer) that interacts with human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, melanoma gangliosides, or a simple transmembrane sequence.

Selective delivery of therapeutic agents to cancer cells in a living body is another area of research where targeting of cancer specific biomarkers is intensively studied. (E. Mastrobattista, G. A. Koning, and G. Storm, “Immonoliposomes for the Targeted Delivery of Antitumor Drugs,” Adv Drug Delivery Reviews 1999, 40: 103-27; J. Sudimack and R. J. Lee, “Targeted Drug Delivery Via Folate Receptor,” Adv Drug Delivery Reviews 2000, 41: 147-62; S. P. Vyas and V. Sihorkar, “Endogenous Carriers and Ligands in Non-Immunogenic Site-Specific Drug Delivery,” Adv Drug Delivery Reviews 2000, 43: 101-64). Immunoliposome-mediated targeting using monoclonal antibodies to folate receptor, (E. Mastrobattista, G. A. Koning, and G. Storm, “Immonoliposomes for the Targeted Delivery of Antitumor Drugs,” Adv Drug Delivery Reviews 1999, 40: 103-27; J. Sudimack and R. J. Lee, “Targeted Drug Delivery Via Folate Receptor,” Adv Drug Delivery Reviews 2000, 41: 147-62) CA-125, (E. Mastrobattista, G. A. Koning, and G. Storm, “Immonoliposomes for the Targeted Delivery of Antitumor Drugs,” Adv Drug Delivery Reviews 1999, 40: 103-27) and HER2/neu antigen (D. B. Kirpotin, J. W. Park, K. Hong, S. Zalipsky, W. L. Li, P. Carter, C. C. Benz, and D. Papahadjopoulos, “Sterically Stabilized anti-HER2 immunoliposomes: design and targeting to human breast cancer cells in vitro,” Biochemistry 1997, 36: 66-75) have been described.

Thus, the herein provided polypeptide can further comprise a tumor specific targeting sequence. For example, the tumor specific targeting sequence can comprise an RGD, NGR, or GSL motif. In one aspect, the tumor specific targeting sequence targets tumor blood vessel endothelial cells. Thus, the polypeptide can comprise the amino acid sequence set forth in SEQ ID NO:11 or SEQ ID NO:12.

6. Effectors

The herein provided compositions can further comprise an effector molecule. By ‘effector molecule’ is meant a substance that acts upon the target cell(s) or tissue to bring about a desired effect. The effect can, for example, be the labeling, activating, repressing, or killing of the target cell(s) or tissue. Thus, the effector molecule can, for example, be a small molecule, pharmaceutical drug, toxin, fatty acid, detectable marker, conjugating tag, nanoparticle, or enzyme.

Examples of small molecules and pharmaceutical drugs that can be conjugated to a targeting peptide are known in the art. The effector can be a cytotoxic small molecule or drug that kills the target cell. The small molecule or drug can be designed to act on any critical cellular function or pathway. For example, the small molecule or drug can inhibit the cell cycle, activate protein degradation, induce apoptosis, modulate kinase activity, or modify cytoskeletal proteins. Any known or newly discovered cytotoxic small molecule or drugs is contemplated for use with the targeting peptides.

The effector can be a toxin that kills the targeted cell. Non-limiting examples of toxins include abrin, modeccin, ricin and diphtheria toxin. Other known or newly discovered toxins are contemplated for use with the provided compositions.

Fatty acids (i.e., lipids) that can be conjugated to the provided compositions include those that allow the efficient incorporation of the peptide into liposomes. Generally, the fatty acid is a polar lipid. Thus, the fatty acid can be a phospholipid. The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

Detectable markers include any substance that can be used to label or stain a target tissue or cell(s). Non-limiting examples of detectable markers include radioactive isotopes, enzymes, fluorochromes, and quantum dots (Qdot®). Other known or newly discovered detectable markers are contemplated for use with the provided compositions.

The effector molecule can be a nanoparticle, such as a heat generating nanoshell. As used herein, ‘nanoshell’ is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells. Nanoshells can be formed with a core of a dielectric or inert material such as silicon, coated with a material such as a highly conductive metal which can be excited using radiation such as near infrared light (approximately 800 to 1300 nm). Upon excitation, the nanoshells emit heat. The resulting hyperthermia can kill the surrounding cell(s) or tissue. The combined diameter of the shell and core of the nanoshells ranges from the tens to the hundreds of nanometers. Near infrared light is advantageous for its ability to penetrate tissue. Other types of radiation can also be used, depending on the selection of the nanoparticle coating and targeted cells. Examples include x-rays, magnetic fields, electric fields, and ultrasound. The problems with the existing methods for hyperthermia, especially for use in cancer therapy, such as the use of heated probes, microwaves, ultrasound, lasers, perfusion, radiofrequency energy, and radiant heating is avoided since the levels of radiation used as described herein is insufficient to induce hyperthermia except at the surface of the nanoparticles, where the energy is more effectively concentrated by the metal surface on the dielectric. The particles can also be used to enhance imaging, especially using infrared diffuse photon imaging methods. Targeting molecules can be antibodies or fragments thereof, ligands for specific receptors, or other proteins specifically binding to the surface of the cells to be targeted.

The effector molecule can be a radioactive isotope. For example, the effector molecule can be a material, such as a “seed” or wire comprising any radioactive isotope suitable for implantation. A number of devices have been employed to implant radioactive seeds into tissues. See, e.g., U.S. Pat. No. 2,269,963 to Wappler; U.S. Pat. No. 4,402,308 to Scott; U.S. Pat. No. 5,860,909 to Mick; and U.S. Pat. No. 6,007,474 to Rydell. In a typical protocol for treating prostate cancer, an implantation device having a specialized needle is inserted through the skin between the rectum and scrotum into the prostate to deliver radioactive seeds to the prostate. The needle can be repositioned or a new needle used for other sites in the prostate where seeds are to be implanted. Typically, 20-40 needles are used to deliver between about 50-150 seeds per prostate. An ultrasound probe can be used to track the position of the needles.

Currently marketed radioactive seeds take the form of a capsule encapsulating a radioisotope. See, e.g., Symmetra.RTM. I-125 (Bebig GmbH, Germany); IoGold.TM. I-125 and IoGold.TM. Pd-103 (North American Scientific, Inc., Chatsworth, Calif.); Best.RTM. I-125 and Best Pd-103 (Best Industries, Springfield, Va.); Brachyseed.RTM. I-125 (Draximage, Inc., Canada); Intersource.RTM. Pd-103 (International Brachytherapy, Belgium); Oncoseed.RTM. I-125 (Nycomed Amersham, UK); STM 1250 I-125 (Sourcetech Medical, Carol Stream, Ill.); Pharmaseed.RTM. I-125 (Syncor, Woodland Hills, Calif.); Prostaseed.TM. I-125 (Urocor, Oklahoma City, Okla.); and I-plant.RTM. I-125 (Implant Sciences Wakefield, Mass.). The capsule of these seeds can be made of a biocompatible substance such as titanium or stainless steel, and be tightly sealed to prevent leaching of the radioisotope. The capsule can be sized to fit down the bore of one of the needles used in the implantation device. Since most such needles are about 18 gauge, the capsule typically has a diameter of about 0.8 mm and a length of about 4.5-mm. The two radioisotopes most commonly used in brachytherapy seeds are iodine (I-¹²⁵) and palladium (Pd-¹⁰³). Both emit low energy irradiation and have half-life characteristics ideal for treating tumors. For example, I-¹²⁵ seeds decay at a rate of 50% every 60 days, so that using typical starting doses their radioactivity is almost exhausted after ten months. Pd-¹⁰³ seeds decay even more quickly, losing half their energy every 17 days so that they are nearly inert after only 3 months.

Radioactive brachytherapy seeds can also contain other components. For example, to assist in tracking their proper placement using standard X-ray imaging techniques, such seeds may contain a radiopaque marker. Markers are typically made of high atomic number (i.e., “high Z”) elements or alloys or mixtures containing such elements. Examples of these include platinum, iridium, rhenium, gold, tantalum, lead, bismuth alloys, indium alloys, solder or other alloys with low melting points, tungsten, and silver. Many radiopaque markers are currently being marketed including: platinum/iridium markers (Draximage, Inc. and International Brachytherapy), gold rods (Bebig GmbH), gold/copper alloy markers (North American Scientific), palladium rods (Syncor), tungsten markers (Best Industries), silver rods (Nycomed Amersham), silver spheres (International Isotopes Inc. and Urocor), and silver wire (Implant Sciences Corp.). Other radiopaque markers include polymers impregnated with various substances (see, e.g., U.S. Pat. No. 6,077,880).

A number of different U.S. patents disclose technology relating to brachytherapy. For example, U.S. Pat. No. 3,351,049 discloses the use of a low-energy X-ray-emitting interstitial implant as a brachytherapy source. In addition, U.S. Pat. No. 4,323,055; U.S. Pat. No. 4,702,228; U.S. Pat. No. 4,891,165; U.S. Pat. No. 5,405,309; U.S. Pat. No. 5,713,828; U.S. Pat. No. 5,997,463; U.S. Pat. Nos. 6,066,083; and 6,074,337 disclose technologies relating to brachytherapy devices.

The effector molecule can be covalently linked to the disclosed peptide. The effector molecule can be linked to the amino terminal end of the disclosed peptide. The effector molecule can be linked to the carboxy terminal end of the disclosed peptide. The effector molecule can be linked to an amino acid within the disclosed peptide. The herein provided compositions can further comprise a linker connecting the effector molecule and disclosed peptide. The disclosed peptide can also be conjugated to a coating molecule such as bovine serum albumin (BSA) (see Tkachenko et al., (2003) J Am Chem Soc, 125, 4700-4701) that can be used to coat the Nanoshells with the peptide.

Protein crosslinkers that can be used to crosslink the effector molecule to the disclosed peptide are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO STAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent) (Methyl-p-hydroxybenzimidate hydrochloride, 98%), DMA (Dimethyladipimidate hydrochloride).

7. Combination Therapies

Provided herein is a composition that comprises the NIP and any known or newly discovered substance that can be administered to a cancer. For example, the provided composition can further comprise one or more of classes of antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone), Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g. Alclofenac; Alclometasone Dipropionate; Algestone Acetonide; alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Morniflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents (e.g. Ethanolamines (like diphenhydramine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

Numerous anti-cancer drugs are available for combination with the present method and compositions. The following are lists of anti-cancer (anti-neoplastic) drugs that can be used in conjunction with the presently disclosed DOCl activity-enhancing or expression-enhancing methods.

Antineoplastic: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 1131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

The herein provide composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

8. Protein Variants

When specific proteins are referred to herein, variants, derivatives, and fragments are contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues, but can occur at a number of different locations at once; insertions usually will be on the order of about from 1 to 10 amino acid residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure unless such a change in secondary structure of the mRNA is desired. Substitutional variants are those in which at least one residue has been removed and a different residue inserted in its place. Such substitutions generally are made in accordance with the following Table 2 and are referred to as conservative substitutions.

TABLE 2
Amino Acid Substitutions

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Pro | Gly |
| Ser | Thr |
| Thr | Ser |
| Trp | Tyr |
| Tyr | Trp; Phe |
| Val | Ile; Leu |

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations shown in Table 2. Conservatively substituted variations of each explicitly disclosed sequence are included within the polypeptides provided herein.

Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

Further information about conservative substitutions can be found in, among other locations, in Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent than the amino acids shown in Table 3. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Biotechnology, 12:678-682 (1994), all of which are herein incorporated by reference at least for material related to amino acid analogs).

Molecules can be produced that resemble polypeptides, but which are not connected via a natural peptide linkage. For example, linkages for amino acids or amino acid analogs can include CH₂NH—, —CH₂S—, —CH₂—CH₂—, CH═CH—(cis and trans), —COCH₂—, CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—5); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. It is understood that peptide analogs can have more than one atom between the bond atoms, such as b-alanine, g-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, greater ability to cross biological barriers (e.g., gut, blood vessels, blood-brain-barrier), and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

It is understood that one way to define any variants, modifications, or derivatives of the disclosed genes and proteins herein is through defining the variants, modification, and derivatives in terms of sequence identity (also referred to herein as homology) to specific known sequences. Specifically disclosed are variants of the nucleic acids and polypeptides herein disclosed which have at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the stated or known sequence. Those of skill in the art readily understand how to determine the sequence identity of two proteins or nucleic acids. For example, the sequence identity can be calculated after aligning the two sequences so that the sequence identity is at its highest level.

Another way of calculating sequence identity can be performed by published algorithms. Optimal alignment of sequences for comparison may be conducted by the local sequence identity algorithm of Smith and Waterman Adv. Appl. Math. 2: 482 (1981), by the sequence identity alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference in their entirety for the methods of calculating sequence identity.

The same types of sequence identity can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

Thus, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of NBS1. Thus, in one aspect, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

9. Nucleic Acids

Also provided are isolated nucleic acids encoding the polypeptides provided herein. For example, disclosed is an isolated nucleic acid encoding a polypeptide having an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Thus, disclosed is an isolated nucleic acid, comprising the nucleic acid sequence set forth in SEQ ID NO:43, SEQ ID NO:44, SEQ ID NO:45, SEQ ID NO:46, SEQ ID NO:47, SEQ ID NO:48, SEQ ID NO:49, or SEQ ID NO:50.

The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U.

By ‘isolated nucleic acid’ or ‘purified nucleic acid’ is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (e.g., a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term ‘isolated nucleic acid’ also refers to RNA, e.g., an mRNA molecule that is encoded by an isolated DNA molecule, or that is chemically synthesized, or that is separated or substantially free from at least some cellular components, e.g., other types of RNA molecules or polypeptide molecules.

Thus, provided is an isolated nucleic acid encoding a polypeptide comprising the amino acid sequence SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Thus, the provided nucleic acid can comprise the nucleic acid sequence SEQ ID NO:43, SEQ ID NO:44, SEQ ID NO:45, SEQ ID NO:46, SEQ ID NO:47, SEQ ID NO:48, SEQ ID NO:49, or SEQ ID NO:50.

The herein provided nucleic acid can be operably linked to an expression control sequence. Also provided is a vector comprising one or more of the herein provided nucleic acids, wherein the nucleic acid is operably linked to an expression control sequence. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

10. Vectors

Provided herein is a vector comprising a nucleic acid encoding the carboxy-terminal amino acid sequence of NBS1, or a conservative variant thereof, wherein the polypeptide does not comprise the full length NBS1. Also provided herein is a vector comprising a nucleic acid encoding the NBS1-binding sequences of ATM, or a conservative variant thereof. For example, the polypeptide can comprise the heat repeat sequences of ATM, or a fragment thereof, that binds NBS1. A preferred vector targets hypoxia tumor tissues. Thus, the vector can be a hypoxia-target adenoviral vector.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as SEQ ID NO:43, SEQ ID NO:44, SEQ ID NO:45, SEQ ID NO:46, SEQ ID NO:47, SEQ ID NO:48, SEQ ID NO:49, or SEQ ID NO:50, into the cell without degradation and include a promoter yielding expression of the gene in the cells into which it is delivered. In some embodiments the promoters are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also disclosed are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. Also disclosed is a viral vector which has been engineered so as to suppress the immune response of the host organism, elicited by the viral antigens. Vectors of this type can carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4, 980, 286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang ‘Generation and identification of recombinant adenovirus by liposome-mediated transfection and PCR analysis’ BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed, and these virons are generated in a cell line such as the human 293 cell line. In one aspect, both the E1 and E3 genes are removed from the adenovirus genome.

Another type of viral vector is based on an adeno-associated virus (AAV). This defective parvovirus can infect many cell types and is nonpathogenic to humans. AAV type vectors can transport about 4 to 5 kb and wild type AAV is known to stably insert into chromosome 19. As an example, this vector can be the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter, which directs cell-specific expression, operably linked to a heterologous gene. Heterologous in this context refers to any nucleotide sequence or gene which is not native to the AAV or B19 parvovirus.

Typically the AAV and B 19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed polypeptides, nucleic acids or vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The compositions can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Promoters controlling transcription from vectors in mammalian host cells may be obtained from various sources, for example, the genomes of viruses such as: polyoma, Simian Virus 40 (SV40), adenovirus, retroviruses, hepatitis-B virus, cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A promoter of this type is the CMV promoter (650 bases). Other such promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. The transcription unit can also contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In certain transcription units, the polyadenylation region is derived from the SV40 early polyadenylation signal and consists of about 400 bases. Transcribed units an contain other standard sequences alone or in combination with the above sequences improve expression from, or stability of, the construct.

The viral vectors can include nucleic acid sequence encoding a marker product. This marker product is used to determine if the gene has been delivered to the cell and once delivered is being expressed. Example marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: Chinese hamster ovary (CHO) DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

11. Adenovirus-Mediated Tumor Gene Therapy

Tumor virotherapy represents a new platform for the treatment of cancer. Appealing features include tumor-selective targeting, and no cross-resistance to current treatments. Human adenoviruses (Ad) in subgroup C (e.g. Ad5) are pathogens causing asymptomatic or mild respiratory infections in young children and induce lifelong immunity. Consequently, adenovirus vectors for tumor gene therapy have attracted considerable interest. Two types of Ad vectors (replication-defective and replication-competent) have been developed for anticancer therapy (Adam and Nasz, 2001; Glasgow et al., 2006). A large number of non-replicating antitumor adenoviral vectors are designed to have a wide variety of tumor-targeting mechanisms, such as immunomodulatory gene therapy, tumor suppressor gene therapy, and chemogene therapy. Replication-competent vectors are designed to replicate in tumor cells selectively, thus causing tumor lyses. Although evidence of antitumor effects has been reported from clinical trials in both types of vectors, efficacies must be improved to obtain substantial clinical benefits. Approaches of integrating gene therapy with conventional radiotherapy have been poorly explored and developed, but offer promises for targeted therapy. Such a combination, utilizing different tumor targeting mechanisms simultaneously, can result in synergistic antitumor effects to generate maximal antitumor effects. Thus, disclosed herein is a hypoxia-driven adenoviral vector which expresses wtNIP in hypoxic tumor tissues. This gene-therapeutic vector has the following two features: 1) tumor-selective and hypoxia-specific targeting, and 2) tumor radiosensitizing.

12. Cells

Also provided is a cell comprising one or more of the herein provided vectors. As used herein, ‘cell’, ‘cell line’, and ‘cell culture’ may be used interchangeably and all such designations include progeny. The disclosed cell can be any cell used to clone or propagate the vectors provided herein. Thus, the cell can be from any primary cell culture or established cell line. The method may be applied to any cell, including prokaryotic or eukaryotic, such as bacterial, plant, animal, and the like. The cell type can be selected by one skilled in the art based on the choice of vector and desired use. The cell can be isolated or in an organism.

Disclosed are animals produced by the process of transfecting a cell within the animal with any of the nucleic acid molecules or vectors disclosed herein. Disclosed are animals produced by the process of transfecting a cell within the animal any of the nucleic acid molecules or vectors disclosed herein, wherein the animal is a mammal. Also disclosed are animals produced by the process of transfecting a cell within the animal any of the nucleic acid molecules or vectors disclosed herein, wherein the mammal is mouse, rat, rabbit, cow, sheep, pig, or primate.

13. Pharmaceutically Acceptable Carrier

The disclosed compositions can be combined, conjugated or coupled with or to carriers and other compositions to aid administration, delivery or other aspects of the inhibitors and their use. For convenience, such composition will be referred to herein as carriers. Carriers can, for example, be a small molecule, pharmaceutical drug, fatty acid, detectable marker, conjugating tag, nanoparticle, or enzyme.

The disclosed compositions can be used therapeutically in combination with a pharmaceutically acceptable carrier. By ‘pharmaceutically acceptable’ is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Thus, provided is a composition comprising one or more of the herein provided polypeptides, nucleic acids, or vectors in a pharmaceutically acceptable carrier. Thus, provided is a composition comprising a combination of two or more of any of the herein provided NBS1 polypeptides in a pharmaceutically acceptable carrier. For example, provided is a composition comprising SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:43, SEQ ID NO:44, SEQ ID NO:45, SEQ ID NO:46, SEQ ID NO:47, SEQ ID NO:48, SEQ ID NO:49, or SEQ ID NO:50 in a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as ‘stealth’ and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The carrier molecule can be covalently linked to the disclosed inhibitors. The carrier molecule can be linked to the amino terminal end of the disclosed peptides. The carrier molecule can be linked to the carboxy terminal end of the disclosed peptides. The carrier molecule can be linked to an amino acid within the disclosed peptides. The herein provided compositions can further comprise a linker connecting the carrier molecule and disclosed inhibitors. The disclosed inhibitors can also be conjugated to a coating molecule such as bovine serum albumin (BSA) (see Tkachenko et al., (2003) J Am Chem Soc, 125, 4700-4701) that can be used to coat microparticles, nanoparticles of nanoshells with the inhibitors.

Protein crosslinkers that can be used to crosslink the carrier molecule to the inhibitors, such as the disclosed peptides, are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BS SS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SLAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent) (Methyl-p-hydroxybenzimidate hydrochloride, 98%), DMA (Dimethyladipimidate hydrochloride).

i. Nanoparticles, Microparticles, and Microbubbles

The term ‘nanoparticle’ refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

For example, the disclosed compositions can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles (Yang, H. H. et al. 2005) and semiconducting nanocrystals (Lin, Y. W., et al. 2006).

The nanoparticle can also be, for example, a heat generating nanoshell. As used herein, ‘nanoshell’ is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells.

Targeting molecules can be attached to the disclosed compositions and/or carriers. For example, the targeting molecules can be antibodies or fragments thereof, ligands for specific receptors, or other proteins specifically binding to the surface of the cells to be targeted.

ii. Liposomes

‘Liposome’ as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (‘MLVs’) and have diameters within the range of 10 nm to 100 μm. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamellar vesicles, which are prepared by more extensive sonication of MLVs, and consist of a single spherical lipid bilayer surrounding an aqueous solution. Unilamellar vesicles (‘ULVs’) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in ‘Liposome Technology’, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (‘OLVs’), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in ‘Liposome Technology’, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the proprotein convertase inhibitors into liposomes. Generally, the fatty acid is a polar lipid. Thus, the fatty acid can be a phospholipid. The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

iii. In vivo/Ex vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### B. METHODS

1. Methods of Treating

Provided herein is a method of increasing the sensitivity of a tissue to radiotherapy, the steps of the method comprising administering to the tissue a composition that inhibits the interaction of NBS1 with ATM, and irradiating the tissue. The tissue can be any tissue for which radiotherapy is desired, including cancer or a benign growth.

i. Cancer

Thus, provided herein is a method of treating cancer in a subject, comprising administering to the cancer a composition that inhibits the interaction of NBS1 with ATM, and irradiating the cancer.

Also provided herein is a method of treating cancer in a subject, comprising administering to the subject a composition that inhibits the interaction of NBS1 with ATM, and administering to the cancer a chemotherapeutic. Thus, the chemotherapeutic of the disclosed method can be, for example, any of the herein disclosed neoplastic drugs.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

a. Head and Neck Cancer

The provided compositions and methods can be used to treat head and neck cancer. In patients with cancer of the head and neck, radiation can be used as primary therapy or as postoperative treatment. Sometimes radiation is given in combination with chemotherapy. One of the most beneficial results of radiotherapy is laryngeal preservation in persons with cancer of the vocal cord. Because of their location, nasopharyngeal cancers are treated primarily with radiation therapy. Many patients can be re-treated successfully should the tumor recur. Postoperatively, patients with large, extensively invasive tumors or tumors that have positive or close margins, and patients with positive lymph nodes are at high risk for local or regional recurrence. Radiation therapy increases the chance of local control of these tumors and often improves survival in patients with tumors of the head and neck.

b. Skin Cancer

The provided compositions and methods can be used to treat skin cancer. Skin cancer can be treated primarily or postoperatively with radiation. Generally, primary treatment is reserved for use in areas where the cosmetic result with surgery may not be suitable. Most commonly, these include areas around the nose, ear, upper lip and commissure, eyelid and canthi. Similarly, postoperative irradiation increases local control in high-risk patients.6

c. Central Nervous System Tumors

The provided compositions and methods can be used to treat cancer of the central nervous system. In these tumors, primary radiotherapy can be indicated because the tumor location precludes surgery. But most commonly, postoperative radiation is employed. Radiation therapy improves survival in many patients with high-grade gliomas and in some patients with low-grade gliomas. New methods of conformal and stereotactic therapy, which more precisely focus the treatment beam using a three-dimensional technique, allow higher doses of radiation to be administered safely and accurately. These techniques can be particularly promising for the treatment of brain tumors.

d. Genitourinary Cancers

The provided compositions and methods can be used to treat genitourinary cancers.

In selected patients with invasive bladder cancer, radiation along with chemotherapy can help to preserve the bladder and its function. Prostate cancer can be treated primarily or postoperatively with radiation. It appears that, stage for stage, radical prostatectomy and primary radiotherapy offer the same chance of disease-free survival for prostate cancer patients. Recently, androgen blockade has been found to enhance local control and improve survival rates. Radiation therapy is associated with lower morbidity in patients with prostate cancer, particularly when conformal therapy or radioactive seed implants are used. With radiation, it is often possible to avoid the occurrence of impotence and incontinence, which are more common with other therapies.

In patients who have had a prostatectomy and who have a high risk of local recurrence, such as those with tumors with positive margins or a rising level of prostate-specific antigen (PSA), radiation can improve local control and survival. Patients with early-stage testicular seminoma have a very high survival rate when treated with low-dose irradiation following radical orchiectomy.

e. Gynecologic Tumors

The provided compositions and methods can be used to treat gynecologic tumors. Early stage (stage I) cervical cancer can be treated with radiotherapy as effectively as with surgery. Later (stages II and III) tumors are best treated with irradiation. Many cervical and endometrial cancers with unfavorable histologic characteristics are better controlled with postoperative radiation. Certain patients with ovarian cancer may benefit from intraperitoneal radioactive phosphorus given postoperatively. Vaginal and vulvar cancers are frequently treated with radiotherapy because the required surgery is often too extensive, and patients are often elderly.

f. Breast Cancer

The provided compositions and methods can be used to treat breast cancer. Radiation has dramatically altered the management of primary breast cancer. Breast conservation, using lumpectomy and radiation therapy, is the treatment of choice in early-stage breast cancer. Cosmetic results are good in most patients, and survival is not compromised. Attempts are being made to identify patients at low risk who can be managed with lumpectomy alone, but so far, all groups of patients have better local control with added radiation. Many patients with locally advanced breast cancer show improvement in local control with radiotherapy, and there is increased survival following radiation.

g. Gastrointestinal Tumors

The provided compositions and methods can be used to treat gastrointestinal tumors. Esophageal cancer is usually advanced by the time the patient seeks treatment. Radiotherapy, along with chemotherapy, appears to be as effective as surgery in most patients with esophageal cancer. In selected patients, preoperative chemoradiotherapy can offer the best results. Patients with stomach cancer also often present with advanced disease. Some studies suggest the best treatment in these patients is postoperative chemoradiotherapy if the tumor is resectable or chemoradiotherapy alone if it is unresectable. Similarly, postoperative chemoradiotherapy or chemoradiotherapy alone is the preferred treatment for resectable and unresectable pancreatic cancer.

Certain high-risk patients with locally advanced colon cancer may have better local control and survival with adjuvant radiation therapy. Preoperative radiotherapy, often administered with chemotherapy, can downstage advanced or low-lying rectal cancers and allow resection and preservation of the sphincter. In high-risk surgical patients, radiation therapy can be helpful postoperatively as well. Anal and perianal cancers are usually treated with combined radiation and chemotherapy because excellent results and sphincter preservation can be obtained.

h. Lung Cancer

The provided compositions and methods can be used to treat lung cancer. Lung cancer should be treated surgically whenever possible. Postoperative radiation improves local control and can improve survival in certain high-risk surgical patients. Unresectable lung cancer can occasionally be made resectable with preoperative radiation. If resection is not possible, an approach combining radiation and chemotherapy is preferred. Administration of chemotherapy may precede or coincide with radiotherapy.

i. Sarcomas

The provided compositions and methods can be used to treat a sarcoma. Wide local excision with preservation of function is indicated for soft tissue sarcomas. High-risk patients who receive preoperative or postoperative irradiation show improvement in local control and survival. By shrinking the tumor, preoperative radiation may allow more limited surgery and improvement in local control, which can also be limb sparing.

j. Lymphomas

The provided compositions and methods can be used to treat a lymphoma. Some patients with either non-Hodgkin's or Hodgkin's lymphoma may be best treated with radiotherapy. Many patients with low-grade non-Hodgkin's lymphoma can be treated with radiation alone, with excellent local control. Some patients with higher-grade non-Hodgkin's lymphoma with stage I or stage II disease have better survival with irradiation after chemotherapy. Hodgkin's lymphoma in selected patients with early-stage disease should be treated with radiation alone or combined with chemotherapy. Radiation therapy may also help control more advanced Hodgkin's lymphomas.

k. Pediatric Cancers

The provided compositions and methods can be used to treat a pediatric cancer. Pediatric cancer patients may benefit from radiation of central nervous system tumors (ependymomas, astrocytomas, medulloblastomas, embryonal tumors, brainstem gliomas, craniopharyngiomas, pineal tumors, cerebellar astrocytomas, optic gliomas, retinoblastomas, spinal cord tumors), neuroblastomas, lymphomas, Ewing's sarcoma, rhabdomyosarcomas and Wilm's tumor.

l. Palliative Care

The provided compositions and methods can be used to for palliative care. In patients with metastatic cancer, radiation often improves quality of life and even survival. Radiation is excellent for relief of painful bone metastases and may prevent pathologic fracture in weight-bearing bones. Generally, the pain relief afforded by radiation allows a reduction in pain medications and drug side effects. Spinal cord compression as a result of cancer is an emergency that can often be treated effectively with radiotherapy alone. Patients with back pain, weakness of the extremities, or problems with bowel or bladder control should be evaluated immediately to rule out cord compression. Superior vena cava syndrome, another potential emergency, usually responds well to radiotherapy. Patients usually present with dyspnea, orthopnea and venous congestion in the neck and upper extremities. Likewise, airway compression caused by cancer can often be treated effectively with radiotherapy. Short courses of radiation may improve median survival and quality of life in patients with brain metastases. If the only clinical disease is a single brain metastasis, the combined approaches of either surgery and whole-brain irradiation or stereotactic radiation and whole-brain irradiation improve the median survival rate most dramatically.

ii. Benign Disease

The provided compositions and methods can be used to treat a benign diseases. For example, radiotherapy can be used to treat Ameloblastoma, Aneurysmal bone cyst, Angiofibroma, Arteriovenous malformation, Chemodectoma, Chordoma, Craniopharyngioma, Desmoid tumor, Graves' opthalmopathy, Gynecomastia associated with hormonal management of prostate cancer, Hemangioma, Heterotopic bone formation, Hypersplenism, Keloid, Keratoacanthoma, Meningioma, Peyronie's disease, Pituitary adenoma, Pterygium, Total lymphoid irradiation for autoimmune disease or organ transplantation, trigeminal neuralgia, thyroid eye disease, and Vascular restenosis prevention.

a. Trigeminal Neuralgia

Thus, provided herein is a method of treating trigeminal neuralgia in a subject, comprising administering to the trigeminal nerve a composition that inhibits the interaction of NBS1 with ATM, and irradiating the trigeminal nerve.

Trigeminal neuralgia is a neuropathic disorder of the trigeminal nerve that causes episodes of intense pain in the eyes, lips, nose, scalp, forehead, and jaw. Trigeminal neuralgia is considered by many to be among the most painful of conditions and once was labeled the suicide disease because of the significant numbers of people taking their own lives before effective treatments were discovered. An estimated one in 15,000 people suffers from trigeminal neuralgia, although numbers may be significantly higher due to frequent misdiagnosis.

The trigeminal nerve is the fifth cranial nerve, a mixed cranial nerve responsible for sensory data such as tactition (pressure), thermoception (temperature), and nociception (pain) originating from the face, above the jawline; it is also responsible for the motor function of the muscles of mastication, the muscles involved in chewing but not facial expression. Several theories exist to explain the possible causes of this pain syndrome. The leading explanation is that a blood vessel is likely to be compressing the trigeminal nerve near its connection with the pons. The superior cerebellar artery is the most-cited culprit. Such a compression can injure the nerve's protective myelin sheath and cause erratic and hyperactive functioning of the nerve. This can lead to pain attacks at the slightest stimulation of any area served by the nerve as well as hinder the nerve's ability to shut off the pain signals after the stimulation ends. This type of injury also may be caused by an aneurysm (an outpouching of a blood vessel); by a tumor; by an arachnoid cyst in the cerebellopontine angle, or by a traumatic event such as a car accident or even a tongue piercing. Two to four percent of patients with TN, usually younger, have evidence of multiple sclerosis, which may damage either the trigeminal nerve or other related parts of the brain. When there is no structural cause, the syndrome is called idiopathic. Postherpetic Neuralgia, which occurs after shingles, may cause similar symptoms if the trigeminal nerve is affected.

The nerve can be damaged to prevent pain signal transmission using a gamma knife or similar radiosurgical device such as Novalis shaped beam. No incisions are involved in this procedure. It uses radiation to bombard the nerve root, this time targeting the selective damage at the same point where vessel compressions are often found. This option is used especially for those people who are medically unfit for a long general anaesthetic, or who are taking medications for prevention of blood clotting (e.g., warfarin).

Thus, the disclosed compositions can be used to radiosensitize the nerve prior to the radiosurgical procedure.

b. Pterygium

Local strontium application can help prevent the local recurrence of a surgically resected eye pterygium. Superficial low-dose radiation treatment can help prevent local recurrence of surgically resected keloids. Radiation therapy is often used to treat pituitary adenomas successfully with minimal morbidity. Low-dose irradiation can sometimes improve Graves' opthalmopathy in selected patients in whom other types of therapy have failed. Likewise, keratoacanthomas that fail to respond to other treatment usually respond well to irradiation. Hemangiomas also respond well to low-dose radiation. Radiotherapy can help control high-risk desmoids and Peyronie's disease. In some postoperative orthopedic patients, low-dose radiation can prevent heterotopic bone formation. Finally, arteriovenous malformations of the central nervous system may be eliminated with stereotactic radiotherapy, if the malformations are not surgically accessible.

Also provided herein is a method of treating pterygium in a subject, comprising administering to the conjunctiva a composition that inhibits the interaction of NBS1 with ATM, and irradiating the conjunctiva.

Pterygium can refer to a benign growth of the conjunctiva. Alternately, it refers to any winglike triangular membrane occurring in the neck, eyelids, knees, elbows, ankles or digits (J Pediatr Orthop B 2004, 13:197-201). An example is popliteal pterygium syndrome, which affects the legs.

When associated with the conjunctiva, a pterygium commonly grows from the nasal side of the sclera. It is associated with, and thought to be caused by ultraviolet-light exposure (e.g. sunlight), low humidity, and dust. The predominance of pterygia on the nasal side is possibly a result of the sun's rays passing laterally through the cornea where it undergoes refraction and becomes focused on the limbic area. Sunlight passes unobstructed from the lateral side of the eye, focusing on the medial limbus after passing through the cornea. On the contralateral side, however, the shadow of the nose medially reduces the intensity of sunlight focused on the lateral/temporal limbus.

Pterygium in the conjunctiva is characterized by elastotic degeneration of collagen and fibrovascular proliferation. It has an advancing portion called the head of the pterygium, which is connected to the main body of the pterygium by the neck. Sometimes a line of iron deposition can be seen adjacent to the head of the pterygium called Stocker's line. The location of the line can give an indication of the pattern of growth. As it is a benign growth, it requires no treatment unless it grows to such an extent that it covers the pupil, obstructing vision. Some patients may also choose surgery if the growth becomes too unsightly. The exact cause is unknown, but it is associated with excessive exposure to wind, sun, or sand. Wearing protective sunglasses with side shields and/or wide brimmed hats and using artificial tears throughout the day may help prevent their formation or stop further growth. For surfers and other water-sport athletes, they should wear eye protection that block 100% of the UV rays from the water.

Occasionally it is found as an incidental finding in middle aged patients who spend a lot of time in the sun. Pterygiums are also among younger men and women who surf, wakeboard, and kiteboard due to excessive exposure to UV rays bouncing off of the water. Skiiers and snowboarders protect their eyes on the snow so athletes participating in water sports also need to take heed of the UV rays and protect their eyes.

While patients can be symptomatically treated w/artificial tears, no reliable medical treatment exists to reduce or even prevent pterygium progression. Definitive treatment is achieved only by surgical removal. Long term follow up is required as pterygium may recur even after complete surgical correction.

c. Thyroid Eye Disease

Also provided herein is a method of treating severe thyroid eye disease in a subject, comprising administering to the eye a composition that inhibits the interaction of NBS1 with ATM, and irradiating the eye.

Thyroid eye disease often occurs in people who develop an overactive thyroid gland. Swelling of the muscles and other tissues in the orbits causes the eyes to become pushed forward and more prominent. The eyes often take on a more staring appearance. In more severe cases the swelling may cause stiffness of the muscles which move the eyes. This can cause a “squint” to develop and may result in double vision. Occasionally the swelling behind the eyeball may press on the nerve from the eye to the brain and disrupt vision. Thyroid eye disease is also called thyroid opthalmopathy, Graves' eye disease or dysthyroid eye disease.

Overactivity of the thyroid gland is usually caused by an “autoimmune condition” This means that cells which normally protect the body from infection develop a “fault” and begin to recognize the thyroid gland as foreign material and attack it. This stimulates the thyroid gland to produce extra thyroid hormones. The attacking process may spill over to the cells behind the eye causing them to swell.

Radiotherapy can be given to the tissues behind the eyeball. It involves usually 10 dosages given over 2 weeks. Two thirds of patients find significant benefit but regrettably one third do not and require other therapy such as orbital decompression. Often this therapy is combined with steroids and immunosuppression.

d. Keloid

Also provided herein is a method of treating keloid scar in a subject, comprising administering to the keloid scar a composition that inhibits the interaction of NBS1 with ATM, and irradiating the keloid scar.

A keloid is a type of scar which results in an overgrowth of tissue at the site of a healed skin injury. Keloids are firm, rubbery lesions or shiny, fibrous nodules and can vary from pink to flesh-colored or red to dark brown in color. A keloid scar is benign, non-contagious and usually accompanied by severe itchiness, sharp pains and changes in texture. In severe cases, it can affect movement of skin. Keloids should not be confused with hypertrophic scars, which are raised scars that do not grow beyond the boundaries of the original wound and may reduce over time.

Keloids expand in claw like growths over normal skin. They have the capability to hurt with a needle-like pain or to itch without warning, although the degree of sensation varies from patient to patient. If the keloid becomes infected, it may ulcerate. The only treatment is to remove the scar completely.

Keloids form within scar tissue. Collagen, used in wound repair, tends to overgrow in this area, sometimes producing a lump many times larger than that of the original scar. Although they usually occur at the site of an injury, keloids can also arise spontaneously. They can occur at the site of a piercing and even from something as simple as a pimple or scratch. They can occur as a result of severe acne or chickenpox scarring, infection at a wound site, repeated trauma to an area, excessive skin tension during wound closure or a foreign body in a wound.

Electron beam radiation can be used at levels which do not penetrate the body deeply enough to affect internal organs. Orthovoltage radiation is more penetrating and slightly more effective. Radiation treatments reduce scar formation if they are used soon after a surgery while the surgical wound is healing.

e. Heterotopic Ossification

Also provided herein is a preventing heterotopic ossification in a subject, comprising administering to the tissue a composition that inhibits the interaction of NBS1 with ATM, and irradiating the tissue.

Heterotopic ossification (HO) is the abnormal formation of true bone within extraskeletal soft tissues. Classically, many diseases sharing this common feature were lumped under the category of myositis ossificans, a term that has fallen into disfavor because primary muscle inflammation is not a necessary precursor and ossification does not always occur in muscle tissue since it frequently shows a predilection for fascia, tendons, and other mesenchymal soft tissues.

Traditionally, various forms of HO have been classified according to the clinical setting and location of the lesion and whether lesions were progressive or isolated occurrences. The term myositis ossificans traumatica is applied to HO occurring after recalled trauma such as blunt injury, surgery, or burns. Logically, the lesion is termed myositis ossificans atraumatica if no inciting trauma can be identified. Lesions have been labeled as panniculitis ossificans when confined to the subcutaneous fat, as rider's bones when found in the adductor muscles, and as shooter's bones when located in the deltoid.

A strong association exists between HO and spinal cord injury, with lesions occurring at multiple sites and showing a strong propensity to recur. Similarly, periarticular HO is seen in patients with traumatic brain injury, with the extent and functional severity of the HO directly related to severity of the intracranial injury. Many other causes of neurologic compromise, including tetanus, poliomyelitis, Guillain-Barré syndrome, and prolonged pharmacologic paralysis during mechanical ventilation, also have been associated with HO formation.

Fibrodysplasia ossificans progressiva (FOP), or Munchmeyer disease, is an autosomal dominant, severely disabling disease resulting in progressive ossification of fascial planes, muscles, tendons, and ligaments. Congenital malformation of the great toes is associated with FOP. HO is a feature of several other diseases, including Albright hereditary osteodystrophy, progressive osseous heteroplasia, and primary osteoma cutis.

HO originates from osteoprogenitor stem cells lying dormant within the affected soft tissues. With the proper stimulus, the stem cells differentiate into osteoblasts and begin the process of osteoid formation, eventually leading to mature heterotopic bone. A variety of bone morphogenetic proteins (BMPs) can stimulate HO when experimentally deposited into soft tissues, suggesting that BMPs play a role in the initiation of HO. A degree of neurologic control is implied but is not well understood.

iii. Radiotherapy

Radiation therapy is a local treatment modality that works by damaging the DNA of malignant cells. Normal cells have a greater ability to repair this damage than tumor cells. Radiation therapy takes advantage of this difference. It is important to note that since damaged cells do not die immediately after treatment, tumors often persist after successful radiation therapy is completed.

Treatment prescriptions are based on the goals of treatment and the potential for side effects. A course of treatment may be as short as one day or as long as 10 weeks, but a typical duration is between two and seven weeks and usually consists of five daily treatments a week. Patients most commonly receive radiation through a linear accelerator, which accelerates electrons to be used as a treatment beam or to generate x-rays to be used as a treatment beam. Treatment is not painful and often lasts less than five minutes.

The goal of treatment may be curative or palliative. If radiotherapy is potentially curative, the length of treatment is often longer and usually consists of smaller daily doses over a longer period of time. This approach minimizes late side effects. If treatment is intended to be strictly palliative, shorter treatment schedules consisting of larger daily treatment doses over a shorter time period are used. In such cases, late side effects are not likely to occur within the patient's lifetime. Furthermore, a shorter treatment program will negatively affect less of the patient's remaining life.

Since radiation is a form of local therapy, side effects are usually limited to the treated area. However, fatigue is one common systemic symptom. Usually, the side effects of radiation are mild, but occasionally they are severe. The side effects can be divided into early and late effects. Early effects occur during or immediately after treatment and typically resolve within three to six weeks following therapy. Late side effects occur months to years after treatment and are often permanent. These effects are the result of tissue injury that leads to necrosis or scarring and, rarely, to carcinogenesis. The occurrence of malignancies secondary to radiation therapy has become well known. While it is true that many genetic factors predispose some patients to second cancers, radiation also contributes to the increased relative risk. The herein disclosed compositions and methods can lessen these side effects by reducing the amount of radiation required.

Radiation therapy has many potential specific indications. It can be given as primary tumor treatment, as pre- or postoperative therapy, or as a component of combination or consolidative therapy.

Radiation therapy is suitable for almost two-thirds of cancer patients and is used for curative and palliative purposes. Many tumors, such as prostate cancer, breast cancer, head and neck cancer, lung cancer, brain tumor, gastro-intestinal tumors, liver cancer, soft tissue sarcomas, cervical cancer, lymphomas etc, will receive radiotherapy as a part of treatment regimen. Radiation therapy includes external beam radiation (such as X-ray, γ-ray, proton and neutron), brachytherapy and radioactive material implementation. Radiation therapy can be administrated by 2-D, 3-D, conformal, intensity-modulated (IMRT) and image-guided (IGRT) approaches. Standard radiotherapy for most of solid tumors is given 2Gy/day with a total dose of around 60Gys (50-70Gys). However, it is routine for the skilled artisan to select the preferred radiation dose based on the specific subject, equipment, and type of tumor. The present method constitutes an improvement on existing radiation therapy methods by increasing the sensitivity of the cancer cells to the radiation. For example, the provided method can result in the cancer cell being at least 30%, 40%, 50%, 60%, 70%, 80%, 90%, 100% or higher sensitivity to the radiation. As used herein, the term ‘sensitivity’ and ‘radiosensitivity’ refer to the number of cells that survive based on a dosage of radiation. Thus, increased radiosensitivity can result in a decrease in the number of cells that survive a dose of radiation, a decrease in the dose of radiation that is required for lethality, or a combination thereof.

Brachytherapy, also known as sealed source radiotherapy or endocurietherapy, is a form of radiotherapy where a radioactive source is placed inside or next to the area requiring treatment. Conversely, external beam radiotherapy, or teletherapy, is the application of radiation that has been externally produced by a linear accelerator. Brachytherapy is commonly used to treat localized prostate cancer and cancers of the head and neck.

Brachytherapy includes Mold brachytherapy, Strontium plaque therapy, Interstitial brachytherapy, Intracavitary brachytherapy, and Intravascular brachytherapy. In mold brachytherapy, superficial tumours can be treated using sealed sources placed close to the skin. Dosimetry is often performed with reference to the Manchester system; a rule-based approach designed to ensure that the dose to all parts of the target volume is within 10% of the prescription dose. Surface Applicator is usually called strontium plaque therapy and is used for very superficial lesions less than 1 mm thick. The plaque is a hollow, thin silver casing that encloses a radioactive Strontium-90 powdered salt. The beta (electron) particles produced from Strontium's radioactive decay have a very shallow penetration. Typically the Sr90 plaque is placed on the bed of a resected pterygium. A stat dose of around 10-12 Gy is delivered by timing the contact. As the electrons only penetrate a few mm of air, radiation protection issues are slightly less but very different to other radiation sources. Cleaning the plaques that are placed on the eye sclera is required but must be gentle because the silver casing is thin and easily damaged. Strontium belongs to the same chemical class as Calcium, i.e., an alkaline earth metal, and so will co-locate in the bone if any strontium salt makes contact with the eye and is absorbed. Operators can prevent exposure to the beta rays by holding the applicator to face away from their bodies. In interstitial brachytherapy, the sources are inserted into tissue. The first treatments of this kind used needles containing Radium-226, arranged according to the Manchester system, but modern methods tend to use Iridium-192 wire. Iridium wire can be arranged either using the Manchester or the Paris system; the latter was designed specifically to take advantage of the new nuclide. Prostate cancer treatment with Iodine-125 seeds is also classified as interstitial brachytherapy. For details of the gamma emitters please see commonly used gamma emitting isotopes. Intracavitary brachytherapy places the sources inside a pre-existing body cavity. The most common applications of this method are gynaecological in nature, although it can also be performed on the nasopharynx. Intravascular brachytherapy places a catheter with the sources inside the vasculature. The most common application of this method is the treatment of coronary in-stent restenosis, although the therapy has also been investigated for use in the treatment of peripheral vasculature stenoses.

High Dose Rate (HDR) brachytherapy is a common brachytherapy method. Applicators in the form of catheters are arranged, usually according to the Manchester or Paris system on, or in the patient. A high dose rate source (often iridium 192, Ir-192) is then driven along the catheters on the end of a wire by a machine while the patient is isolated in a room. The source dwells in a preplanned position for a preset time before stepping forward along the catheter and repeating, to build up the required dose distribution. The advantage of this treatment over implanting radioactive sources directly is that there is lower staff exposure and the source can be more active due to low staff exposure, thus making treatment times quicker.

Just like High dose rate (HDR), Low dose rate (LDR) involves implanting radioactive material and can be implanted temporarily or permanently. LDR brachytherapy with a machine works in a similar way. Another variant is the sources being in the form of active and inactive balls which are again, driven into the patient using a machine.

In some aspects, the disclosed method can further comprise administering to the healthy tissue of the subject a radioprotectant. Generally, radioprotectants will comprise compositions that scavenge free-radicals and prevent oxidative damage.

iv. Chemotherapy

The majority of chemotherapeutic drugs can be divided in to: alkylating agents, antimetabolites, anthracyclines, plant alkaloids, topoisomerase inhibitors, monoclonal antibodies, and other antitumour agents. All of these drugs affect cell division or DNA synthesis. Some newer agents don't directly interfere with DNA. These include the new tyrosine kinase inhibitor imatinib mesylate (Gleevec® or Glivec®), which directly targets a molecular abnormality in certain types of cancer (chronic myelogenous leukemia, gastrointestinal stromal tumors). In addition, some drugs can be used which modulate tumor cell behaviour without directly attacking those cells. Hormone treatments fall into this category of adjuvant therapies.

The chemotherapeutic of the disclosed method can be an alkylating agent. Alkylating agents are so named because of their ability to add alkyl groups to many electronegative groups under conditions present in cells. Cisplatin and carboplatin, as well as oxaliplatin are alkylating agents. Other agents are mechloethamine, cyclophosphamide, chlorambucil. They work by chemically modifying a cell's DNA.

The chemotherapeutic of the disclosed method can be an anti-metabolite. Anti-metabolites masquerade as purine ((azathioprine, mercaptopurine)) or pyrimidine—which become the building blocks of DNA. They prevent these substances becoming incorporated in to DNA during the ‘S’ phase (of the cell cycle), stopping normal development and division. They also affect RNA synthesis. Due to their efficiency, these drugs are the most widely used cytostatics.

The chemotherapeutic of the disclosed method can be a plant alkaloids or terpenoids. These alkaloids are derived from plants and block cell division by preventing microtubule function. Microtubules are vital for cell division and without them it can not occur. The main examples are vinca alkaloids and taxanes.

The chemotherapeutic of the disclosed method can be a vinca alkaloid. Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine, Vinblastine, Vinorelbine, Vindesine, and Podophyllotoxin. Podophyllotoxin is a plant-derived compound used to produce two other cytostatic drugs, etoposide and teniposide. They prevent the cell from entering the G1 phase (the start of DNA replication) and the replication of DNA (the S phase). The exact mechanism of its action still has to be elucidated. The substance has been primarily obtained from the American Mayapple (Podophyllum peltatum). Recently it has been discovered that a rare Himalayan Mayapple (Podophyllum hexandrum) contains it in a much greater quantity, but as the plant is endangered, its supply is limited. Studies have been conducted to isolate the genes involved in the substance's production, so that it could be obtained recombinantively.

The chemotherapeutic of the disclosed method can be a taxane. The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

The chemotherapeutic of the disclosed method can be a topoisomerase inhibitor. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include the camptothecins irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

The chemotherapeutic of the disclosed method can be an antitumour antibiotic (Antineoplastics). The most important immunosuppressant from this group is dactinomycin, which is used in kidney transplantations.

The chemotherapeutic of the disclosed method can be an (monoclonal) antibody. Monoclonal antibodies work by targeting tumour specific antigens, thus enhancing the host's immune response to tumour cells to which the agent attaches itself. Examples are trastuzumab (Herceptin), cetuximab, and rituximab (Rituxan or Mabthera). Bevacizumab is a monoclonal antibody that does not directly attack tumor cells but instead blocks the formation of new tumor vessels.

The chemotherapeutic of the disclosed method can be a hormonal therapy. Several malignancies respond to hormonal therapy. Strictly speaking, this is not chemotherapy. Cancer arising from certain tissues, including the mammary and prostate glands, may be inhibited or stimulated by appropriate changes in hormone balance. Steroids (often dexamethasone) can inhibit tumour growth or the associated edema (tissue swelling), and may cause regression of lymph node malignancies. Prostate cancer is often sensitive to finasteride, an agent that blocks the peripheral conversion of testosterone to dihydrotestosterone. Breast cancer cells often highly express the estrogen and/or progesterone receptor. Inhibiting the production (with aromatase inhibitors) or action (with tamoxifen) of these hormones can often be used as an adjunct to therapy. Gonadotropin-releasing hormone agonists (GnRH), such as goserelin possess a paradoxic negative feedback effect followed by inhibition of the release of FSH (follicle-stimulating hormone) and LH (luteinizing hormone), when given continuously. Some other tumours are also hormone dependent, although the specific mechanism is still unclear.

The composition of the disclosed methods can comprise a peptide that inhibits the interaction of NBS1 with ATM, e.g., an isolated polypeptide or a nucleic acid encoding a polypeptide comprising a carboxy-terminal amino acid sequence of NBS1, or a conservative variant thereof (e.g., NIP) as described herein. The composition of the method can comprise an isolated polypeptide or a nucleic acid encoding a polypeptide comprising the NBS1-binding sequences of ATM, or a conservative variant thereof. For example, the polypeptide can comprise the heat repeat sequences of ATM, or a fragment thereof, that binds NBS1.

In one aspect, the polypeptide can be any polypeptide comprising the carboxy-terminal most amino acids of NBS1. Thus, the provided polypeptide can comprise the C-terminal-most 4 to 30 amino acids of NBS1, including the C-terminal most 4, 5, 6, 7, 8, 9, 10, 11, 12, 13, 14, 15, 16, 17, 18, 19, 20, 21, 22, 23, 24, 25, 26, 27, 28, 29, 30 amino acids of NBS1, or a fragment thereof. For example, the provided polypeptide can comprise amino acids 734 to 754 of NBS1 (SEQ ID NO:1). The provided polypeptide can comprise a conservative amino acid substitution within the C-terminal-most 4 to 30 amino acids, including amino acids 734 to 754, of NBS1 (SEQ ID NO:1). In this context, the peptide can comprise 1, 2 or 3 conservative amino acid substitutions. The polypeptide can comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The polypeptide can comprise an amino acid sequence with at least 95% sequence identity to SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

In an alternative aspect, the polypeptide does not comprise the C-terminal most 1, 2, 3, 4, 5, 6, 7, 8, 9, or 10 amino acids, for example.

In a further aspect, the polypeptide can be any polypeptide comprising the NBS1-binding domain of ATM. Thus, the herein provided polypeptide can be any polypeptide comprising heat repeats 2 and/or 7 of ATM. Thus, the herein provided polypeptide can be any polypeptide comprising amino acids 248-522 of ATM (SEQ ID NO:51). Thus, the herein provided polypeptide can be any polypeptide comprising SEQ ID NO:56. Thus, the herein provided polypeptide can be any polypeptide comprising amino acids 1436-1770 of ATM (SEQ ID NO:51). Thus, the herein provided polypeptide can be any polypeptide comprising SEQ ID NO:57. The provided polypeptide can comprise a conservative amino acid substitution within the heat repeats 2 and/or 7 of ATM. In this context, the peptide can comprise 1, 2 or 3 conservative amino acid substitutions. The polypeptide can comprise an amino acid sequence with at least 95% sequence identity to amino acids 248-522 of ATM (SEQ ID NO:51) or amino acids 1436-1770 of ATM (SEQ ID NO:51). The polypeptide can comprise an amino acid sequence with at least 95% sequence identity to SEQ ID NO:56. The polypeptide can comprise an amino acid sequence with at least 95% sequence identity to SEQ ID NO:57.

2. Screening Methods

Disclosed herein is a method of identifying a radiosensitizing agent, comprising contacting a sample comprising NBS1 and ATM polypeptides with a candidate agent, and detecting the interaction between the NBS1 and ATM polypeptides, a decrease in the interaction between the NBS1 and ATM polypeptides as compared to controls indicating the candidate agent is radiosensitizing. The method is in one aspect a screening assay, such as a high-throughput screening assay. Thus, the contacting step can be in a cell-based or cell-free assay. For example, the interaction between the NBS1 and ATM polypeptides can be detected using fluorescence polarization. Thus, the NBS1 and/or ATM polypeptide can comprise a fluorophore. The herein disclosed NBS1 polypeptide can be used as a positive control.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on NBS1-ATM interaction should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits NBS1-ATM interaction. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions.

3. Methods of Administration

The compositions may be administered topically, orally, or parenterally. For example, the compositions can be administered extracorporeally, intracranially, intravaginally, intraanally, subcutaneously, intradermally, intracardiac, intragastric, intravenously, intramuscularly, by intraperitoneal injection, transdermally, intranasally, or by inhalant. As used herein, ‘intracranial administration’ means the direct delivery of substances to the brain including, for example, intrathecal, intracisternal, intraventricular or trans-sphenoidal delivery via catheter or needle.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

As used herein, ‘topical intranasal administration’ means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as ‘stealth’ and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8, from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels (e.g., poloxamer gel), drops, suppositories, sprays, liquids and powders. Conventional pharmaceutical carriers, aqueous, powder or oily bases, thickeners and the like may be necessary or desirable. The disclosed compositions can be administered, for example, in a microfiber, polymer (e.g., collagen), nanosphere, aerosol, lotion, cream, fabric, plastic, tissue engineered scaffold, matrix material, tablet, implanted container, powder, oil, resin, wound dressing, bead, microbead, slow release bead, capsule, injectables, intravenous drips, pump device, silicone implants, or any bio-engineered materials.

In one aspect the provided pharmaceutically acceptable carrier is a poloxamer. Poloxamers, referred to by the trade name Pluronics®, are nonionic surfactants that form clear thermoreversible gels in water. Poloxamers are polyethylene oxide-polypropylene oxide-polyethylene oxide (PEO-PPO-PEO) tri-block copolymers. The two polyethylene oxide chains are hydrophilic but the polypropylene chain is hydrophobic. These hydrophobic and hydrophilic characteristics take charge when placed in aqueous solutions. The PEO-PPO-PEO chains take the form of small strands where the hydrophobic centers would come together to form micelles. The micelle, sequentially, tend to have gelling characteristics because they come together in groups to form solids (gels) where water is just slightly present near the hydrophilic ends. When it is chilled, it becomes liquid, but it hardens when warmed. This characteristic makes it useful in pharmaceutical compounding because it can be drawn into a syringe for accurate dose measurement when it is cold. When it warms to body temperature (when applied to skin) it thickens to a perfect consistency (especially when combined with soy lecithin/isopropyl palmitate) to facilitate proper inunction and adhesion. Pluronic® F127 (F127) is widely used because it is obtained easily and thus it is used in such pharmaceutical applications. F127 has a EO:PO:EO ratio of 100:65:100, which by weight has a PEO:PPO ratio of 2:1. Pluronic gel is an aqueous solution and typically contains 20-30% F-127. Thus, the provided compositions can be administered in F127.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual doctor in the event of any counterindications. Dosage can vary, and can be administered in one or more dose administrations daily, for one or several days. Guidance can be found in the literature for appropriate dosages for given classes of pharmaceutical products. The range of dosage largely depends on the application of the compositions herein, severity of condition, and its route of administration.

For example, in applications as a laboratory tool for research, the NBS1 peptide compositions can be used in doses as low as 0.01% w/v. The dosage can be as low as 0.02% w/v and possibly as high as 2% w/v in topical treatments. Significantly higher concentrations of the compositions by themselves or in combination with other compounds may be used in applications like cancer/tumor therapy or as an early concentrated bolus immediately following an acute tissue injury. Thus, upper limits of the provided polypeptides may be up to 2-5% w/v or v/v if given as an initial bolus delivered for example directly into a tumor mass. Recommended upper limits of dosage for parenteral routes of administration for example intramuscular, intracerebral, intracardicardiac and intraspinal could be up to 1% w/v or v/v depending on the severity of the injury. This upper dosage limit may vary by formulation, depending for example on how the polypeptide(s) is combined with other agents promoting its action or acting in concert with the polypeptide(s).

For continuous delivery of the provided polypeptides, for example, in combination with an intravenous drip, upper limits of 0.01 g/Kg body weight over time courses determined by the doctor based on improvement in the condition can be used. In another example, upper limits of concentration of the provided nucleic acids delivered topically would be 5-10 μg/cm² depending for example on how the nucleic acid is combined with other agents promoting its action or acting in concert with the nucleic acids. This would be repeated at a frequency determined by the Doctor based on improvement. In another example, upper limits of concentration of the provided nucleic acids delivered internally for example, intramuscular, intracerebral, intracardicardiac and intraspinal would be 50-100 μg/ml of solution. Again, the frequency would be determined by the Doctor based on improvement.

Also disclosed is the pre-conditioning of an area with the provided polypeptides prior to surgery. The concentration of the polypeptides can be 10-200 μM mixed in with 10-30% pluronic gel or any such carrier that enables penetration of the peptide(s) within the site of interest for a period of at least 3-6 hours prior to surgery. This pre-procedural conditioning can improve the subsequent healing response to surgery, including reduced inflammatory response.

Viral vectors remain highly experimental tools that nonetheless show considerable potential in clinical applications. As such, caution is warranted in calculation of expected dosage regimes for viral vectors and will depend considerably on the type of vector used. For example, retroviral vectors infect dividing cells such as cancer cells efficiently, intercalating into the host cell genome and continuing expression of encoded proteins indefinitely. Typical dosages of retroviruses in an animal model setting are in the range of 10⁷ to 10⁹ infectious units per ml. By contrast, adenoviruses most efficiently target post-mitotic cells, but cells are quickly eliminated by the host immune system or virus is eventually lost if infected cells resume proliferation and subsequently dilute the viral episomal DNA. Indeed, this transient time course of infection may be useful for short-term delivery of the composition described herein in certain clinical situations, for example in amelioration of a small injury. In animal models, concentrations of 10⁸-10¹¹ infectious units per ml of adenovirus are typical for uses in research. Dose ranges of vectors based on data derived from animal models would be envisaged to be used eventually in clinical setting(s), pending the development of pharmaceutically acceptable formulation(s).

Following administration of a disclosed composition, such as a polypeptide, for promoting radiosensitization, the efficacy of the therapeutic composition can be assessed in various ways well known to the skilled practitioner. For instance, one of ordinary skill in the art will understand that a composition, such as a polypeptide, disclosed herein is efficacious in promoting radiosensitization in a subject by observing that the composition can reduce scar tissue formation, reduce fibrotic tissue formation, improve tissue regeneration, or reduce inflammation in the subject following tissue injury. Methods for measuring these criteria are known in the art and discussed herein.

4. Methods of Making the Compositions

The compositions disclosed herein and the compositions necessary to perform the disclosed methods can be made using any method known to those of skill in the art for that particular reagent or compound unless otherwise specifically noted.

For example, the provided nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1 Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

One method of producing the disclosed polypeptides, such as SEQ ID NO:3, SEQ ID NO:4, SEQ ID NO:5, SEQ ID NO:6, SEQ ID NO:7, SEQ ID NO:8, SEQ ID NO:9, and SEQ ID NO:10, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a protein, or fragment thereof. (Grant Ga. (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Disclosed are processes for making the compositions as well as the intermediates leading to the compositions. There are a variety of methods that can be used for making these compositions, such as synthetic chemical methods and standard molecular biology methods. It is understood that the methods of making these and the other disclosed compositions are specifically disclosed. Disclosed are nucleic acid molecules produced by the process comprising linking in an operative way a nucleic acid encoding a polypeptide disclosed herein and a sequence controlling the expression of the nucleic acid. Disclosed are cells produced by the process of transforming the cell with any of the herein disclosed nucleic acids. Disclosed are any of the disclosed peptides produced by the process of expressing any of the herein disclosed nucleic acids. Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate. Also disclose are animals produced by the process of adding to the animal any of the cells disclosed herein.

### C. USES

The disclosed compositions can be used in a variety of ways as research tools. For example, the disclosed compositions, such an isolated polypeptide comprising SEQ ID NOs:3, 4, 5, 6, 7, 8, 9, and 10 can be used to study the interactions between NBS1 and ATM, by for example acting as inhibitors of binding. Other uses are disclosed, apparent from the disclosure, and/or will be understood by those in the art. Other uses are disclosed, apparent from the disclosure, and/or will be understood by those in the art.

### D. DEFINITIONS

It must be noted that as used herein and in the appended claims, the singular forms ‘a,’ ‘an, ’ and ‘the’ include plural reference unless the context clearly dictates otherwise. Thus, for example, reference to ‘a peptide’ includes a plurality of such peptides, reference to ‘the peptide’ is a reference to one or more peptides and equivalents thereof known to those skilled in the art, and so forth.

‘Optional’ or ‘optionally’ means that the subsequently described event, circumstance, or material may or may not occur or be present, and that the description includes instances where the event, circumstance, or material occurs or is present and instances where it does not occur or is not present.

Ranges can be expressed herein as from ‘about’ one particular value, and/or to ‘about’ another particular value. When such a range is expressed, another embodiment includes from the one particular value and/or to the other particular value. Similarly, when values are expressed as approximations, by use of the antecedent ‘about,’ it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as ‘about’ that particular value in addition to the value itself. For example, if the value ‘10’ is disclosed, then ‘about 10’ is also disclosed. It is also understood that when a value is disclosed that ‘less than or equal to’ the value, ‘greater than or equal to the value’ and possible ranges between values are also disclosed, as appropriately understood by the skilled artisan. For example, if the value ‘10’ is disclosed the ‘less than or equal to 10’ as well as ‘greater than or equal to 10’ is also disclosed. It is also understood that the throughout the application, data is provided in a number of different formats, and that this data, represents endpoints and starting points, and ranges for any combination of the data points. For example, if a particular data point ‘10’ and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word ‘comprise’ and variations of the word, such as ‘comprising’ and ‘comprises, means ‘including but not limited to,’ and is not intended to exclude, for example, other additives, components, integers or steps.

As used herein, ‘inhibit,’ ‘inhibiting, and ‘inhibition’ mean to decrease an activity, response, condition, disease, or other biological parameter. This can include, but is not limited to, the complete loss of activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### E. EXAMPLES

### 1. Example 1

### Characterization of an NBS1 C-Terminal Peptide That Can Inhibit Ataxia Telangiectasia Mutated (ATM)-Mediated DNA Damage Responses and Enhance Radiosensitivity

### i. Materials and Methods

Cell culture: Human tumor cell lines HeLa and DU-145 (ATCC, Manassas, Va.), and human SV-40 transformed fibroblast cell line GM9607 (Corriell Cell Repositories, Camden, N.J.) were maintained in exponential growth in DMEM-10% FBS, in a 5% CO₂ humidified atmosphere. The glioma cell line M059J (Corriell Cell Repositories) were maintained in exponential growth in RPMI-15% FBS, in a 5% CO₂ humidified atmosphere.

Peptides synthesis: All peptides were synthesized by Abgent (San Diego, Calif.) and labeled with a biotin tag at their N-terminus for detection in vitro. Three peptides were produced, one containing the polyarginine (R₉) internalization sequence alone, and a wild-type NBS1 inhibitory peptide (wtNIP) corresponding to amino acids 735-744 of human NBS1, and a random sequence peptide in which a.a. 735-744 of human NBS1 were scrambled (scNIP). The peptides were dissolved in DMSO, stored at −20° C., and reconstituted in DMEM-10% FBS prior to use.

Irradiation: An X-RAD 320 Irradiation Cabinet (Precision X-ray, East Haven, Conn.) was employed at 320 KV and 160 mA, with a 0.8 mm Sn+0.25 mm Cu+1.5 mm Al (HVL≅3.7 Cu) filter at a TSD of 20 cm and a dose rate of 3.4Gy/min. All irradiations were conducted under normal atmospheric pressure and temperature.

Immunoprecipitation and Western blotting: For co-immunoprecipitation of ATM, NBS1 and MRE11, cells were lysed 1 hour in ice-cold lysis buffer, which consisted of 10 mM Tris-HCl (pH 7.5), 100 mM NaCl, 5 mM EDTA, 0.5% NP-40, 5 mM Na₃VO₄, 1 mM NaF, and 1 mM PMSF. After centrifugation, supernatants were incubated with indicated antibodies. After extensive washing with the lysis buffer, immunoprecipitates were analyzed by immunoblot using specific antibodies. For western blotting analysis, samples (cell lysates or immunoprecipitates) were separated on 4-12% SDS-poly-acrylamide gels, transferred to nitrocellulose membranes, and probed with various antibodies.

Immunofluorescence microscopy: Exponentially growing cultures of cells were plated on sterile, 22 cm² coverslips, and incubated for 24 hours at 37° C. in 5% CO₂ humidified air before they are treated with the NIP peptides at room temperature. Coverslips were washed with PBS and fixed with 4% paraformaldehyde-0.25% Triton X-100 for 15 minutes at room temperature, blocked for 30 minutes at room temperature, and incubated with FITC-conjugated streptavidin or anti-γH2AX and phospho-NBS1 antibodies (Rockland Immunochemicals, Gilbertsville, Pa.) for 1 hour at room temperature. Coverslips were then mounted with Vectashield Elite (Vector Labs, Burlingame, Calif.) and observed with a Leica fluorescence microscope. Images were captured at 40× magnification using a Q Imaging Retiga Exi digital camera and analyzed with Image Pro-Plus 5.1 software.

MTT assay: For cytotoxicity studies, exponentially growing cultures of HeLa or DU-145 cells were harvested, plated in 96-well plates (5000 cells/well) in complete media, and incubated overnight. On the following day cells were treated with the NIP peptides (0, 5, 10, 20, 50 or 100 μM) or Taxol (0, 10, 50 or 100 uM) as a positive control. At the end of the time course, an MTT cell viability assay (Promega Corp., Madison, Wis.) was used according to manufacturer's guidelines to determine peptide cytotoxicity.

Colony formation assays: To determine the radiosensitivity, the colony forming assay was incorporated. Cells were harvested with 0.125% trypsin-0.05% EDTA, pelleted and re-suspended in 1 ml fresh media with a 22 g needle to disperse clumps prior to hemocytometer counting in trypan blue. Cells were then plated at limiting dilutions in E-well plates and allowed to adhere overnight. Cultures were treated with PBS, R₉, wtNIP, or scNIP for 1 hour, and irradiated (0-6Gy). Fresh peptides were added every four hours until 24 hours after IR, when the medium was replaced with peptide-free medium. Cultures were incubated for 10-12 days, harvested and stained with 0.5% crystal violet in methanol. Colony number was determined with a dissecting microscope. A population of >50 cells was counted as one colony, and the number of colonies was expressed as a percentage of the value for untreated mock irradiated control cells. The surviving curves were plotted by linear regression analyses and the D₀ value represents the radiation dose that leads to 37% of survival. To determine the radiosensitizing potential of the peptides in comparison to other small molecule inhibitors, the sensitizing enhancement ratio (SER) was calculated based on the dose of radiation required to reduce survival to 37% in the presence of scNIP or wtNIP. The following formula was used:

$$SER = \frac{{D}_{0} for scNIP treated cells}{{D}_{0} for wtNIP treated cells}$$

Statistics: To establish statistical significance, Student's t-test was incorporated. The data were first fit to each experimental group over a dose range of 0-6Gy. Significant differences were established at p<0.05.

### i. Results

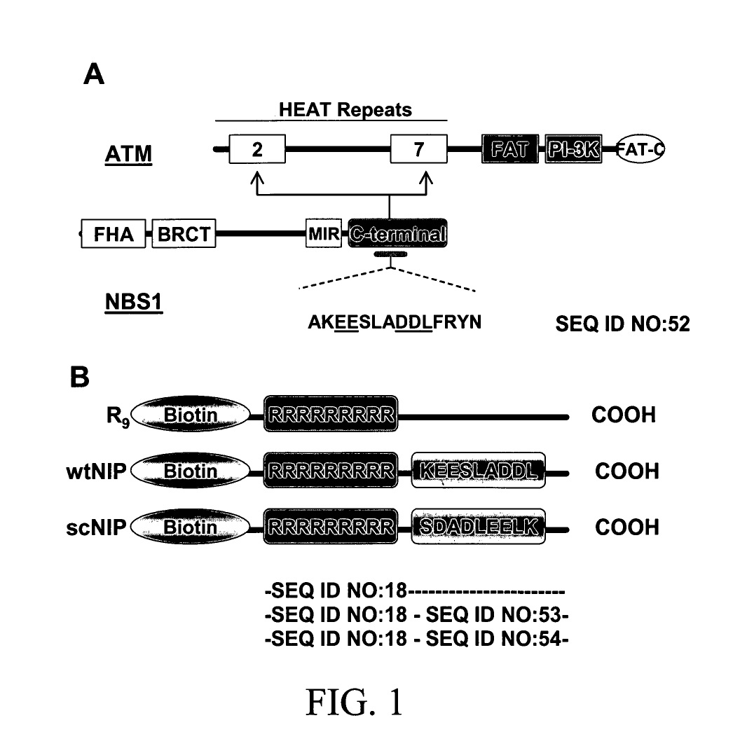

Internalization and Cytotoxicity of the C-Terminal NBS1 Inhibitory Peptides (NIP fusion protein): The C-terminal NBS1 domain is critical for its binding to ATM, and an NBS1 truncated derivative lacking the C-terminal 20 residues does not associate with ATM in vitro (Cerosaletti and Concannon, 2003, 2004; Falck et al., 2005; Cerosaletti et al., 2006). In addition, expression of an NBS1 transgene lacking the ATM binding domain in NBS cells leads to a dramatic reduction in ATM activation (Difilippantonio et al., 2005). Because inhibiting NBS1 association with ATM leads to suboptimal ATM activation after IR, the NBS1-ATM interaction can be a novel target for developing radiosensitizers. One approach to inhibiting NBS1-ATM interaction is to use small peptides containing the conserved C-terminal sequence, which can compete with endogenous NBS1-ATM interactions (FIG. 1A). Therefore, peptides were designed containing two functional domains: one an interfering domain that will inhibit the NBS1-ATM association, and the other an internalization domain that will transport the interfering peptides into cells. For the interfering domain, the amino acid sequences containing the conserved C-terminal motif of NBS1 was used as shown in FIG. 1B. This sequence contains the shortest ATM binding motif based on in vitro data. For the internalization domain, a the polyarginine (R₉) sequence was used. The polyarginine sequence has been shown to have a significant efficiency of transporting small peptides and proteins across the plasma membrane (Fuchs and Raines, 2004; Deshayes et al., 2005). Three peptides were generated, including the R₉-alone, and a wtNIP corresponding to amino acids 73 to 44 of human NBS1. The third peptide was designed as a negative control, using a random sequence generator to produce a peptide in which amino acids 735 to 744 of NBS1 were scrambled (scNIP). These peptides were labeled with a biotin tag at their N termini for detection in vitro.

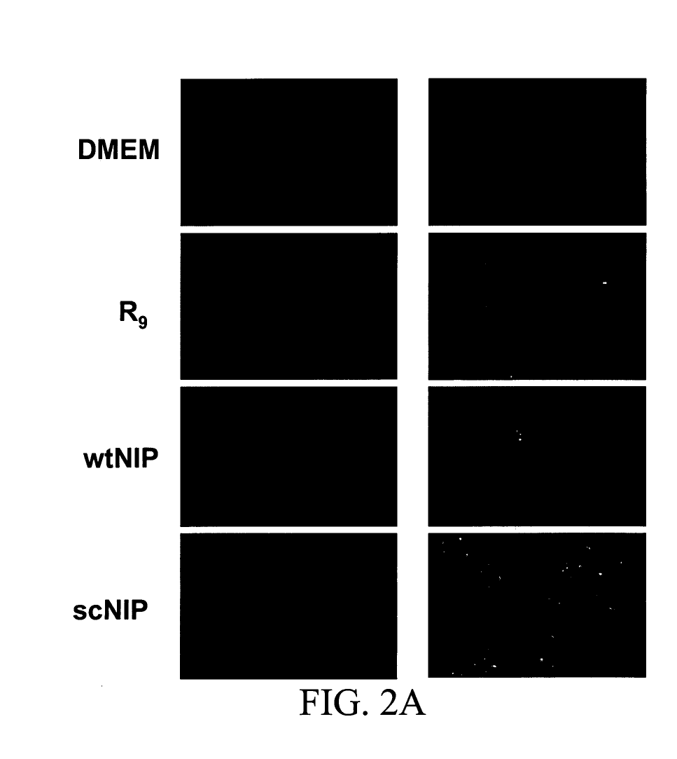

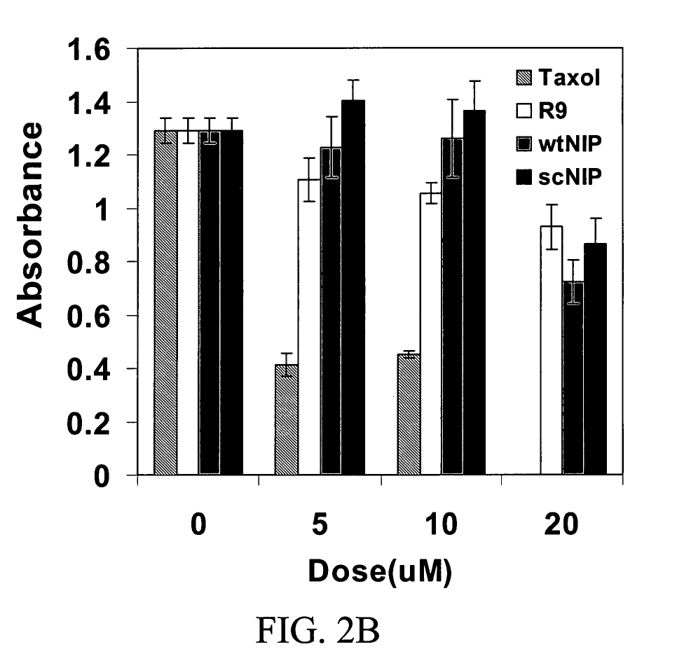

First, the internalization of the peptides was evaluated. Cells treated with the peptide were probed with a fluorescein-conjugated streptavidin antibody to determine the presence of the biotinylated peptides. Treatment of HeLa cells with R₉, wtNIP, or scNIP at a concentration of 10 μM for 1 h led to a significant cellular uptake of peptide (FIG. 2). R₉, wtNIP, and scNIP internalization was localized to the cytoplasmic and nuclear compartments, whereas the control group, treated with DMEM alone, showed no fluorescent signal.

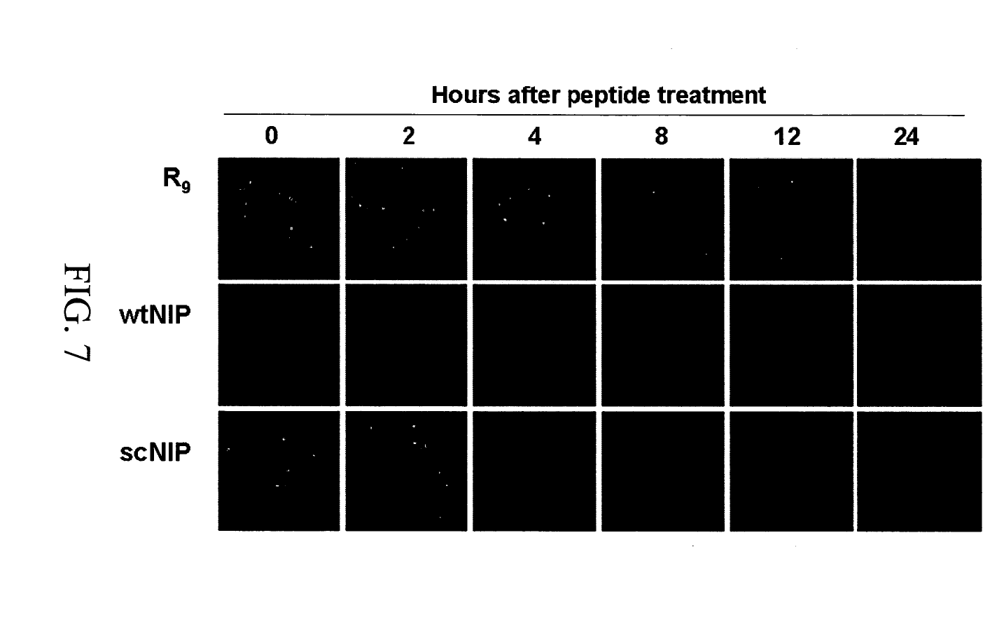

Because the peptides would be used in radiation studies, the length of time the peptides remain in cells was determined to ensure that the peptides would be present throughout the DNA repair process after IR. As shown in FIG. 7, immediately after incubation with the peptides, all sample groups showed distinct presence of peptides. Within 2 hours of treatment, cells continued to display a strong distribution of R₉, wtNIP and scNIP but fluorescent intensity levels of wtNIP and scNIP began to decrease within 4 hours with a substantial decline by 8 hrs. R₉ remained slightly elevated at 8 hours while wtNIP and scNIP intensities were much weaker. This is in an agreement with literature which suggests that R₉ sequences translocate easier and remain present longer than when they are not coupled to a molecule or peptide (Jones et al., 2005). By 12 hours, cells treated with R₉ peptides still displayed prominent staining, while cells treated with wtNIP or scNIP showed much weaker cytoplasmic staining with no observed nuclear staining. Within 24 hours, cells treated with R₉ showed cytoplasmic staining, but the nuclear signal was no longer visible, cells treated with wtNIP or scNIP showed no detectable presence of the peptides. These data indicate that the NIP peptide can stay in cells for at least 4 hours. These data indicate that the NIP peptides could be added to cells at time 0, then every 4 to 6 h in the first 24 h after treatment with IR to achieve maximum inhibitory effects.

Next, in vitro cytotoxicity of R₉, wtNIP and scNIP was determined. HeLa cells grown in 96-well plates were treated with the peptides (0, 5, 10, 20, 50, or 100 μM) or paclitaxel (0, 10, 20, 50, or 100 μM) for 24 h. After treatment, the MTT assay was used to measure the production of solubilized formazan, a metabolic indicator of cell proliferation. The peptides demonstrated no growth inhibitory or cytotoxic effects up to 72 h after treatment (FIG. 2B), when the peptide doses were lower than 20 μM. Based on the cytotoxicity observed in the MTT assay, 10 μM was chosen as the working concentration for all subsequent experiments. The effect of 10 μM R₉, wtNIP, and scNIP on clonogenic survival displayed no significant difference between treatment groups (p<0.05). It is noteworthy that dose and time course experiments have been preformed in several other cell lines, and the data confirmed rapid internalization and minimal cytotoxicity of these peptides.

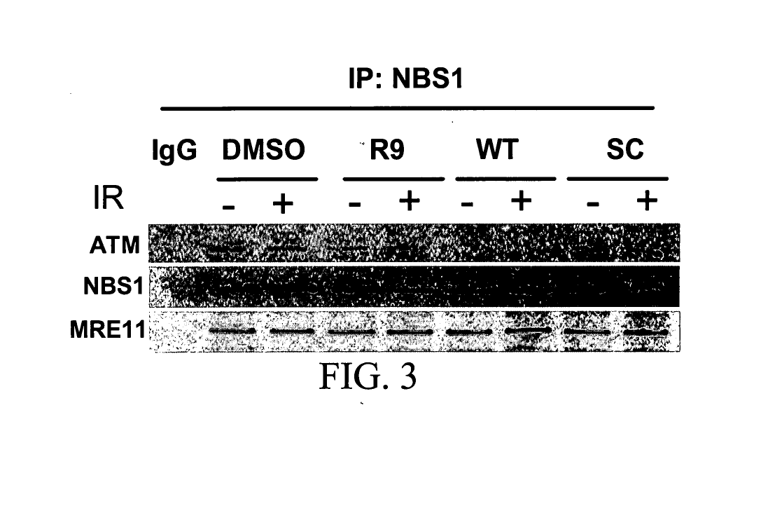

wtNIP Abrogated the NBS1-ATM Interaction: To investigate whether R₉-conjugated NIP peptides could inhibit NBS1-ATM interactions, coimmunoprecipitation experiments were performed in cells treated with the NIP peptides. Four hours after peptide treatment, HeLa cells were harvested and subjected to immunoprecipitation using an anti-NBS1 antibody. The immunoprecipitates were then blotted with anti-ATM, NBS1, and MRE11 antibodies. A normal level of ATM-NBS1 association was observed in R₉-treated cells compared with control cells. However, in wtNIP-treated cells, NBS1 was no longer able to bring down ATM (FIG. 3). Furthermore, the wtNIP affected only the NBS1-ATM interaction and did not interfere with NBS1 binding to MRE11. In contrast, scNIP did not affect the NBS1-ATM interaction. In cells treated with IR, wtNIP showed an effect similar to that in unirradiated cells. These observations demonstrate that wtNIP can abrogate the NBS1-ATM interaction in the absence or the presence of DNA damage.

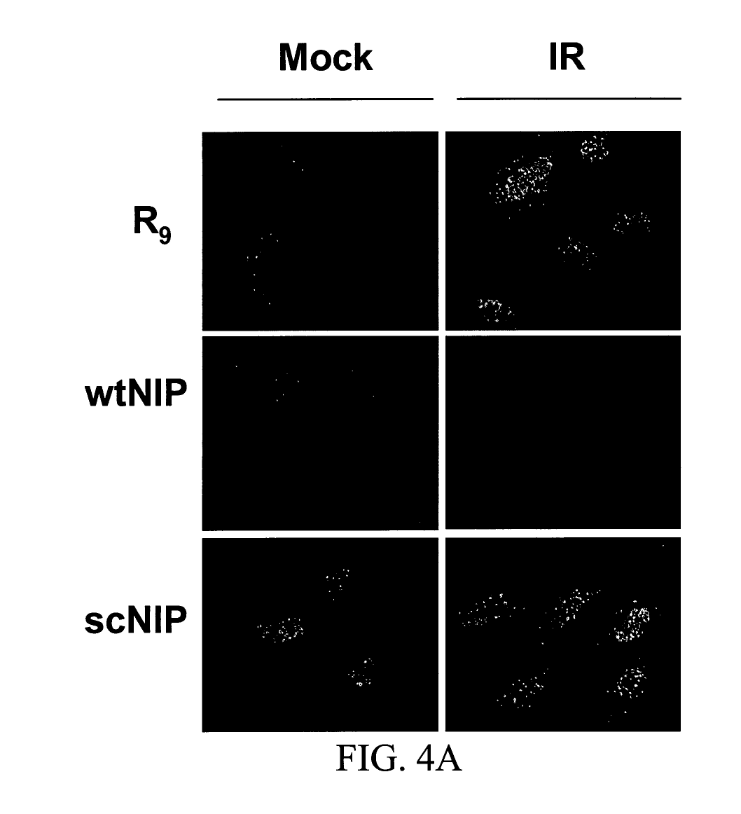

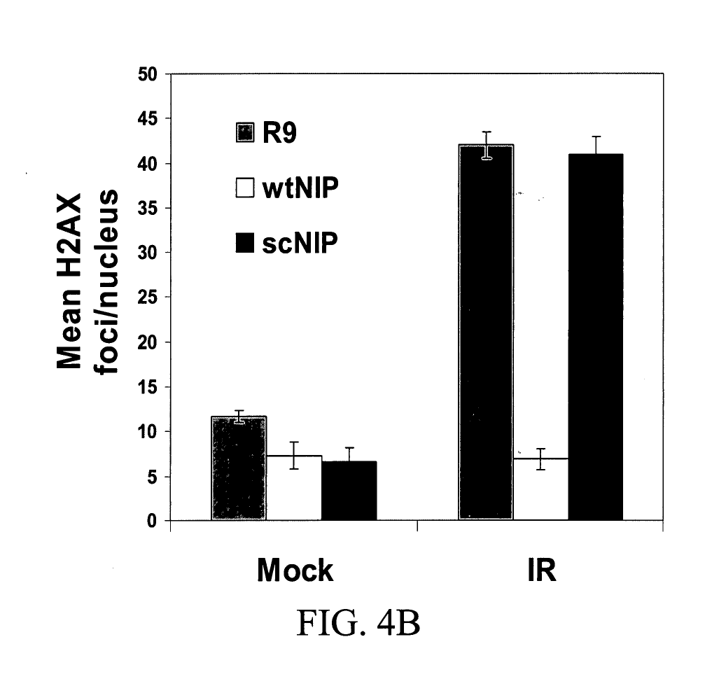

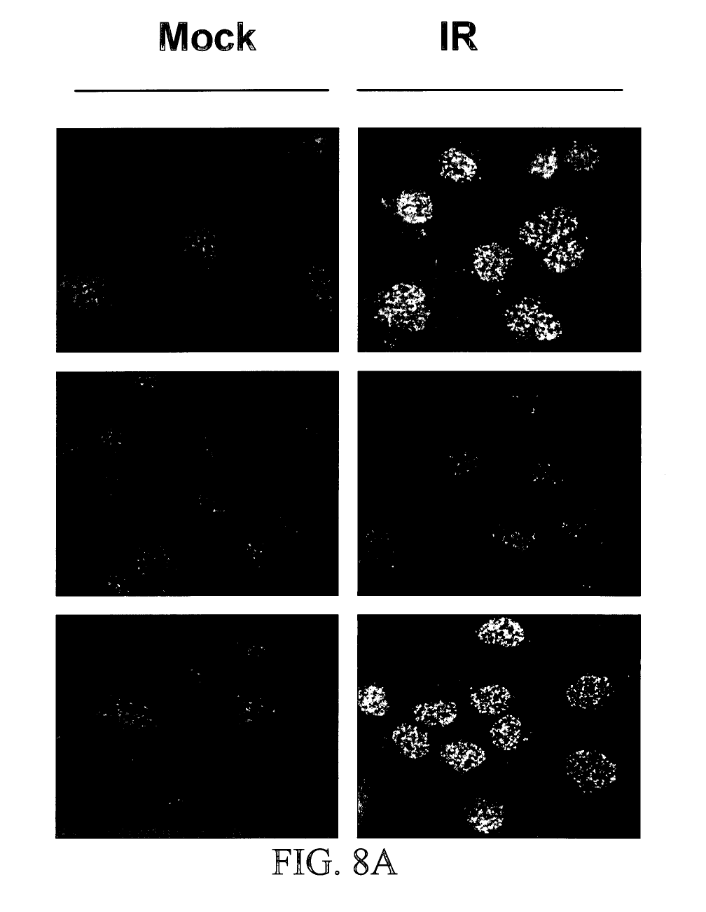

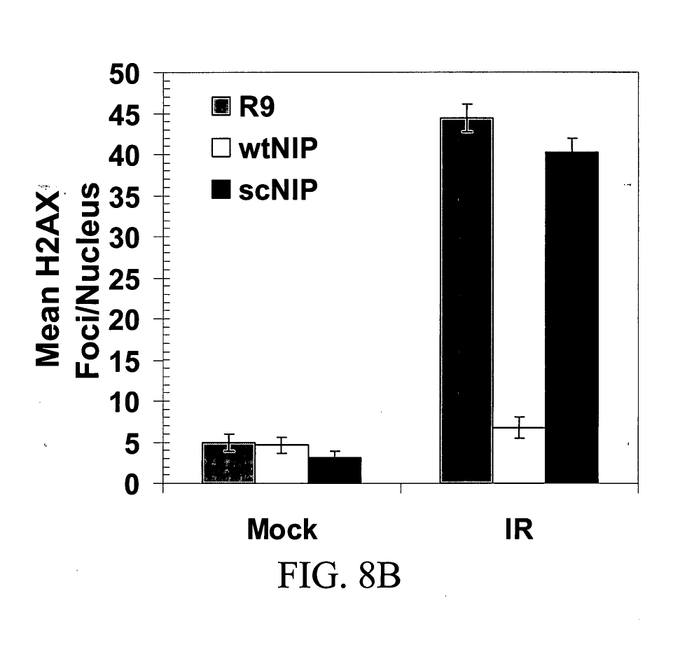

wtNIP Inhibits IR-Induced γ-H2AX and NBS1 pSer343 Focus Formation: One of the earliest responses to IR-induced DNA damage is the formation of γ-H2AX foci, which requires functional ATM (Burma et al., 2001; Furuta et al., 2003). Because wtNIP showed an inhibitory effect on the NBS1-ATM interaction, it was investigated whether IR-induced γ-H2AX focus formation was inhibited by the peptide. Immunofluorescence microscopy was used to detect the presence of γ-H2AX foci in mock-irradiated or irradiated cells in the presence of R₉, wtNIP or scNIP. While R₉ showed no significant inhibition of γ-H2AX foci formation, wtNIP can inhibit γ-H2AX foci formation 30 minutes after treatment with 6Gy IR (FIG. 4). The average number of γ-H2AX foci/nucleus in HeLa cells significantly increased after IR in cells treated with R₉ (42 foci/nucleus) or scNIP (41 foci/nucleus), whereas cells treated with wtNIP displayed only an average of 6.9 γ-H2AX foci/nucleus, similar to that of mock-irradiated cells (FIG. 4). Similar results were observed in DU-145 cells, whereas R₉ or scNIP exposure did not affect IR-induced focus formation, and wtNIP showed significantly reduced H2AX foci/nucleus (FIG. 8). Therefore, IR-induced γ-H2AX focus formation can be inhibited by wt-NIP.

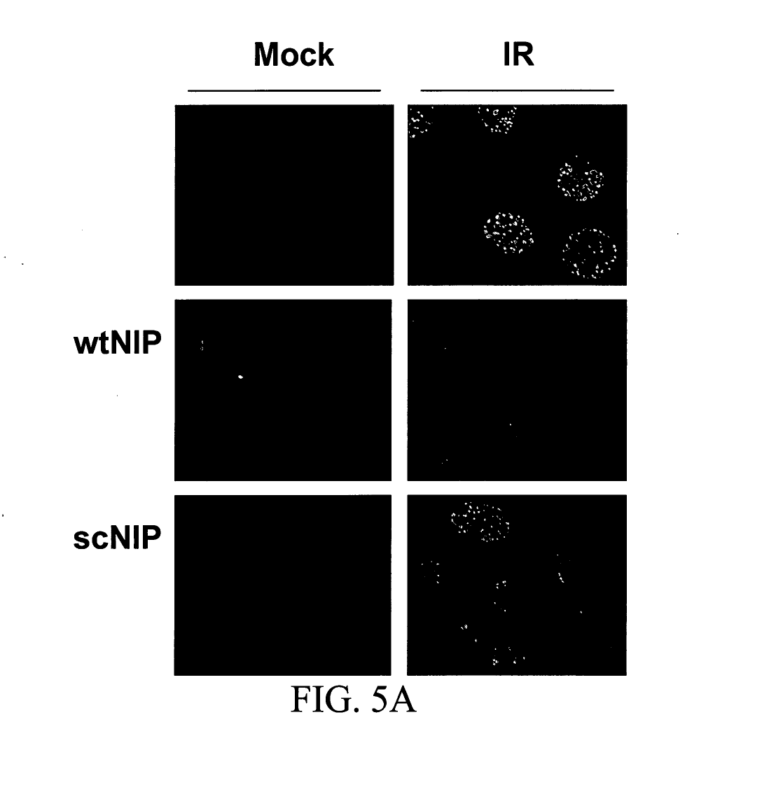

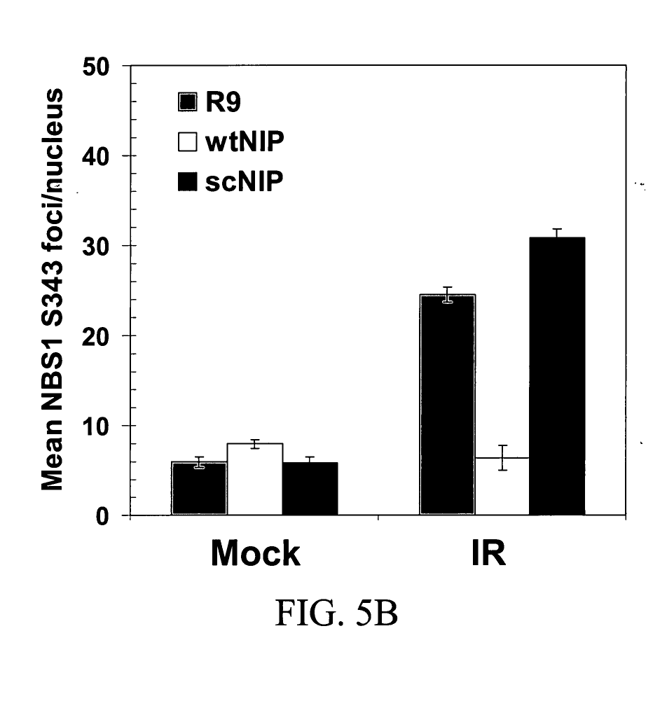

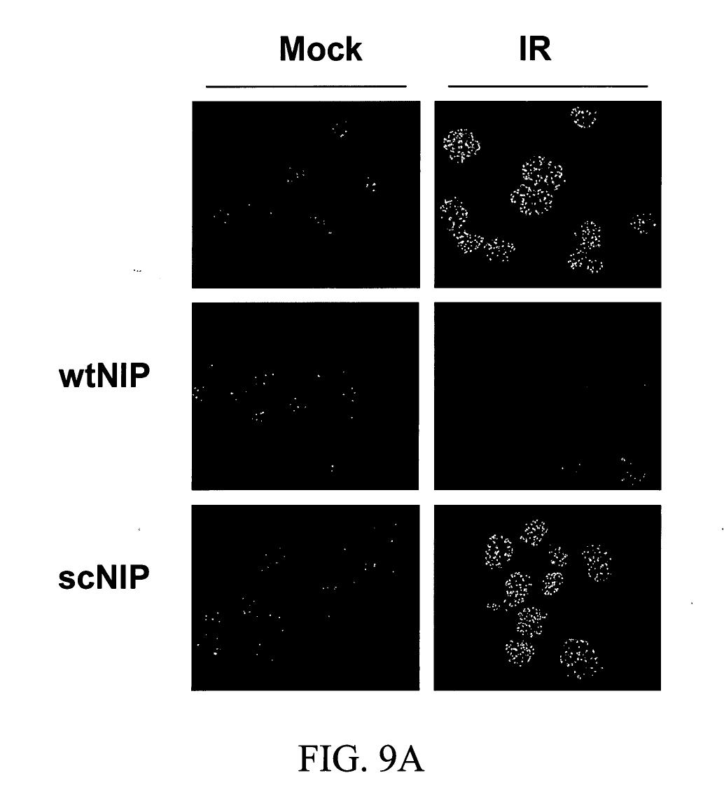

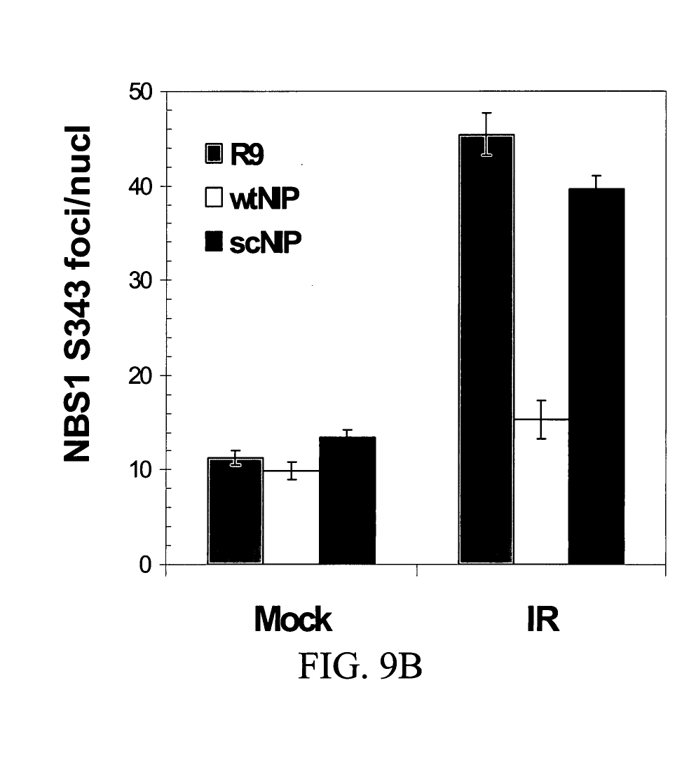

To further support the idea that wtNIP can inhibit ATM-mediated DNA damage pathways, IR-induced NBS1 focus formation, an event considered to be an ATM-dependent process at the sites of DSBs (Lim et al., 2000) was investigated. NBS1 foci are a result of ATM-mediated NBS1 phosphorylation on serine 343. Using an anti-phospho-Ser343 NBS1 antibody, it was observed that NBS1 phosphorylation was significantly inhibited in cells treated with wtNIP compared with those treated with R₉ or scNIP (FIGS. 5A and 9A). The average number of foci in mock-irradiated HeLa cells was 6, 8, and 6 for R₉, wtNIP, and scNIP, respectively. Cells treated with R₉ or scNIP displayed 25 and 31 foci per nucleus, whereas cells treated with wtNIP showed only 6 foci per nucleus after treatment with 6-Gy IR (FIG. 5B).

It is important to note that there was a low level of background focus formation for both NBS1 and γ-H2AX phosphorylation, which has been correlated to mitosis in normally growing mammalian cell cultures (McManus and Hendzel, 2005).

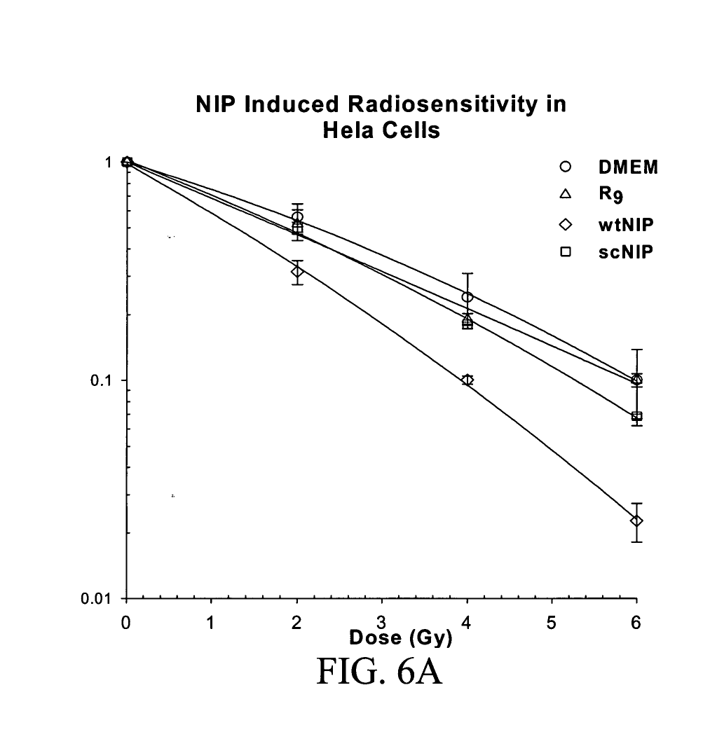

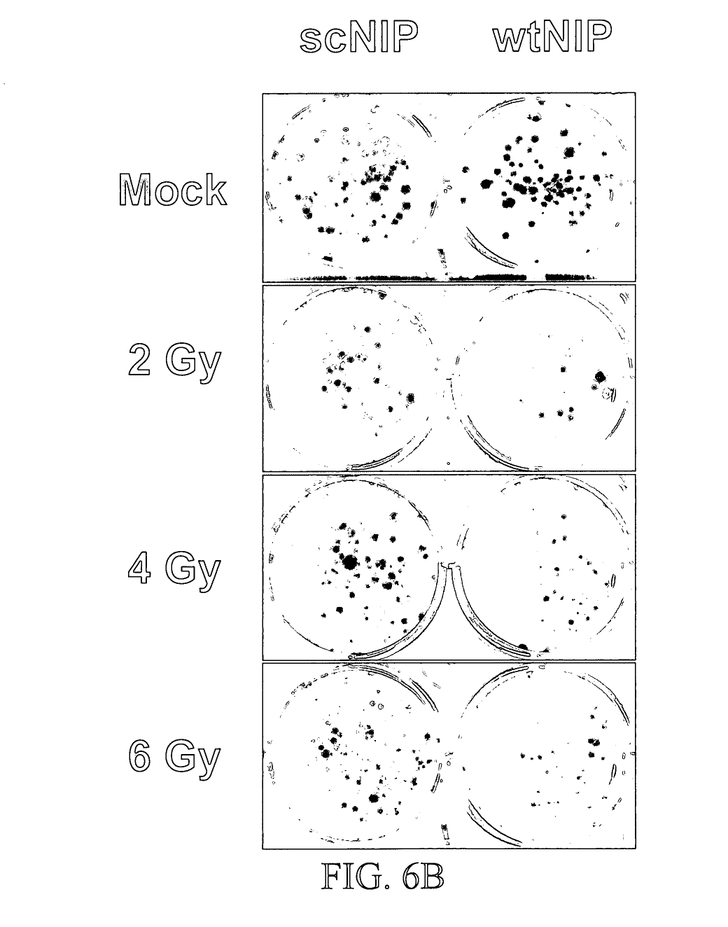

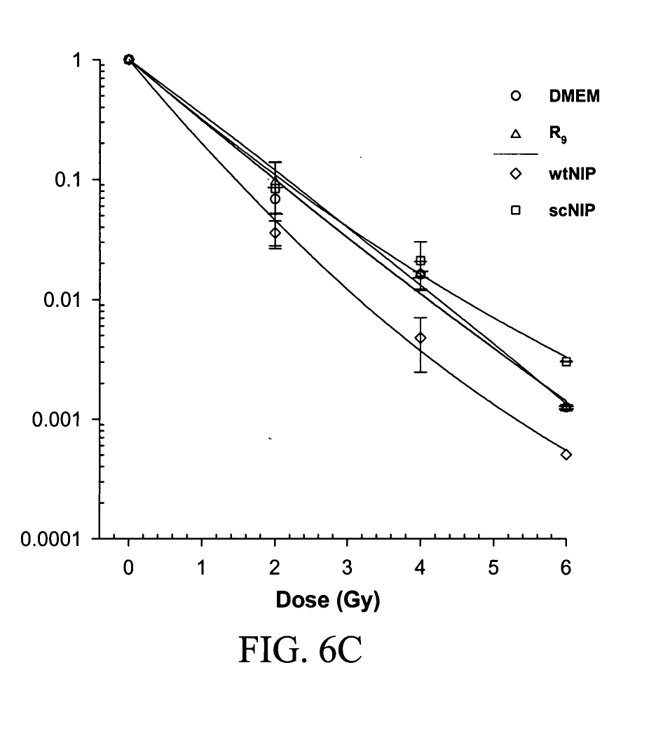

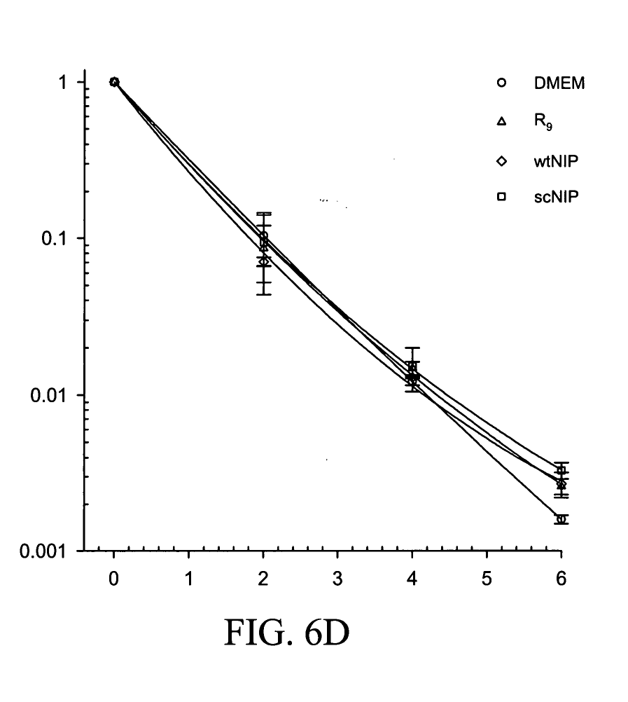

wtNIP Increases Radiation Sensitivity: Whether exposure to the NIP peptides will increase cellular radiosensitivity was then tested using the colony forming assay. FIG. 6A depicts the survival curves for HeLa cells treated with R₉, wtNIP, or scNIP over a dose range of 0 to 6 Gy. Neither R₉ nor scNIP affects radiosensitivity, whereas wtNIP can significantly decrease IR-induced survival.

After treatment with 2Gy, the survival of cells treated with wtNIP was 31.4% compared to 52% and 49.7% for R₉ and scNIP treated cells. At 4Gy, survival of cells treated with wtNIP decreases to 4.5% compared to 11.8% and 11.2% for R₉ and scNIP treated cells. A dose increase to 6Gy lead to a modest decline in survival of cells treated with wtNIP, 1.7%, compared to 5.3% and 6.9% for cells treated with R₉ or scNIP, respectively. The sensitizer enhancement ratio, the relative effectiveness of the enhancer, for wtNIP treated cells was 1.66, 2.61, and 3.12 at 2, 4, and 6Gy respectively.

Radiation survival curves were characterized based on Dₒ to define the effect of NIP effect on radiosensitivity. D₀ represents the mean lethal dose required for 37% survival and is a measure of the intrinsic radiosensitivity of the cell. D₀ values for HeLa treated with wtNIP were 1.9 compared with 3.0 for cells treated with scNIP. To establish the statistical significance of wtNIP-induced radiosensitivity, Student's t test (paired two-sample for means) was incorporated. The data were first fit to each experimental group over a dose range of 0 to 6 Gy. Significant differences (p<0.05) in clonogenic survival were observed between cells treated with wtNIP and those treated with DMEM, R₉, or scNIP. The SER was 1.58. This is comparable with other tested radiosensitizers, including gemcitabine, 5-fluorouracil, pentoxifylline, vinorelbine, and some ATM-specific radiosensitizers with SERs from 1.1 to 2.5 (Zhang et al., 1998; Lawrence et al., 2001; Robinson and Shewach, 2001; Strunz et al., 2002; Collis et al., 2003; Zhang et al., 2004). These observations have been confirmed in the prostate cancer cell line DU-145 with an SER of 1.46. Taken as a whole, they provide strong evidence for the radiosensitizing potential of the wtNIP peptide.

Because wtNIP contains the conserved ATM binding sequence of NBS1, and this sequence is also conserved in the C terminus of ATR-interacting protein and KU80, the interacting proteins of ATR and DNA-PKcs, respectively, it could also inhibit ATR or DNA-PKcs (Abraham, 2001). To test this possibility, colonyforming assays were performed in cell lines with defective ATM (GM9607) or DNA-PKcs (M059J). Although treatment with wtNIP led to an increase in radiosensitivity in M059J cells (FIG. 6C) with an SER of 1.83, GM9607 (FIG. 6D) displayed no change in radiosensitivity. Because GM9607 cells are ATM-deficient and have functional ATR and DNA-PKcs, these observations strongly indicate that wtNIP can specifically target ATM, but not ATR or DNA-PKcs, to achieve radiosensitization.

### 2. Example 2

### Animal Studies

The in vivo radiosensitizing effect of these peptides is tested on mouse tumor xenograft models and a zebrafish embryo model. Tumor-targeting NIP peptides that can specifically accumulate in tumor cells are administered. In addition to confirming the radiosensitizing effect of the wtNIP peptide and conservative variants thereof on mouse breast cancer and prostate cancer xenograft models, the following questions are addressed: 1) how do the NIPS specifically radiosensitize tumor cells; 2) does the peptide also radiosensitize normal tissues; 3) how does the tumor micro-vessel density affect the radiosensitizing enhancement ratio; and 4) what is the radiosensitizing effect of the wtNIP peptide on zebrafish embryos?

i. The Mouse Model

The time course necessary to get appropriate level of NGR (a tumor homing motif) conjugated fusion peptides to the xenografts is determined. The effect of the radiosensitizing peptides on xenografts grown in mice is then determined. To accomplish this, human breast and prostate tumor xenografts are developed in mice, the mice are injected with the peptides, and the peptides are allowed to target the xenograft cells for the appropriate length of time.

NGR—the Tumor Homing Motif: The polyarginine sequence can achieve efficient internalization for the fused NIP peptides. However, another approach to achieve this goal is to utilize sequences that have both internalization and tumor specific targeting abilities. One such peptide is the NGR motif which includes the cyclic tumor-homing peptide, CNGRC (SEQ ID NO:11). The NGR-containing peptides have proven useful for delivering cytotoxic drugs, pro-apoptotic peptides, and the tumor necrosis factor α to tumor vasculature (Ellerby et al., 1999; Arap et al., 1998; Arap et al., 2002; Curnis et al., 2002). More interestingly, it has been shown that NGR peptides can bind to prostatic primary and metastatic tumors, but not to normal prostate tissues (Pasqualini et al., 2000). Therefore the NGR sequence is used for the animal studies. Three NGR-NIP fusion peptides are synthesized, including NGR-only, NGR-wtNIP, and NGR-scNIP. The NGR sequence have been successfully utilized, demonstrating tumor homing and internalization abilities.

Establishment of MCF-7 and PC-3 xenografts: Establishment of the MCF-7 breast cancer and the PC-3 prostate cancer xenografts is performed. Specific pathogen-free, 4-6 week old male nu/nu (nude) mice are obtained and housed in sterilized filter-topped cages kept in laminar flow isolators. Mice are fed autoclaved food and water ad libitum. Mice are acclimated for one week prior to use in study protocols. All procedures involving the animals are performed under sterile conditions in a laminar flow hood. MCF-7 or PC-3 tumor cells (2×10⁶ per mouse in PBS) are injected s.c. into the flanks of athymic nude mice. In all experiments, tumors are allowed to establish and grow before any treatment is initiated.

In vivo distribution of the peptides: Once tumors reach approximately 100 mm³, animals are randomized and treated with the NGR-only, NGR-wtNIP, or NGR-scNIP peptides at doses ranging from 0.5-2 mg/kg by one of two routes: intraperitoneal (ip) or intra-tumoral injection (it). 0, 6, 12, or 24 hours after injection, the mice are euthanized, and the tumor tissue and normal tissues surrounding the tumor tissue is obtained. Whole blood is isolated up to 24 hours after peptide injection. The samples are assessed by a flow-activated cell sorting (FACS) analysis when stained with an FITC-conjugated streptavidin antibody. Splenic cells are analyzed by performing a splenectomy up to 24 hours after peptide injection of the mice. These experiments provide information on how fast the peptides can reach the tumor tissue, how long they will remain in the tumors, and whether the peptides will also accumulate in normal tissues. Localization of the peptides within tumor tissues is analyzed by dissection of the tumor tissues after up to 24 hours after peptide injection. The tumor tissue specimen is stained with FITC-conjugated streptavidin, and immunofluorescence microscopy is performed.

Delivery of radiation: Xenografts are implanted into the flanks of mice through s.c. injections of 0.1 ml of PBS containing 2×10⁶ human MCF-7 or PC-3 carcinoma cells using a 23G needle. Once the tumors reach 100 mm³, the mice are randomized and injected with peptides via ip or it. Dose and time for peptide exposure are determined. For intraperitoneal injection, the volumes of peptides is less than 20 ml/Kg. For intratumoral injection, the volume is less than 10 ml/kg. Following a short interval to allow peptides to target tumors, mice are transported from the animal center in filter top cages to the radiation room. The irradiator unit is a Precision X-RAD 320 Irradiation System. The dose rate for the irradiator at the distance of 50 cm is 2.8Gy/min, while at the distance of 25 cm is 5.6 Gy/min. Both single dose (10 or 20Gy) and fractionated dose (2Gy×5 times or 2Gy×10 times) are performed. The interval for the fractionated radiation is 24 hours and irradiation duration is less than 4 minutes. During the radiation procedure, mice are briefly (less than 5 minutes) restrained in a Plas Labs (Lansing, Mich.) clear plastic mouse-restraining device (tube) to allow the tumors to be targeted by radiation.

Measurement of tumor radiation response: After radiation, tumor volumes are monitored twice-weekly for no more than 8 additional weeks using dial calipers. Tumors are not allowed to grow past 1000 mm³ or to erupt or ulcerate through the skin. Mice are euthanized if any of these endpoints are reached. Tumor growth is reported as an average tumor volume, calculated as π*(w*l*h)/2, where w is width, 1 is length, and h is height in mm. Tumor volume as a function of time is plotted to compare the sensitizing effect of the peptides after radiotherapy.

Measurement of normal tissue response: NGR-wtNIP peptide tends to specifically target tumor cells, and a critical endpoint for evaluating this therapeutic agent to be useful in the clinic is how it affects the normal tissue radiation response. Radiation response is investigated on early (skin) and late (lung) responding normal tissues. These tissues were chosen because they fall into radiation fields for many cancer types, especially breast cancer radiotherapy. The second reason that they were chosen is that there are established methods and standards to evaluate skin and lung radiation response. Mice are treated with the NGR-fusion peptides before 10Gy radiation is delivered. To study skin damage, radiation is given locally to the right rear foot on restrained, non anaesthetized mice. To avoid using anesthetics (which may influence blood flow), fixing of the leg in the correct position for treatment is achieved by applying a small drop of histoacrylic glue to the restraining jig in the region of the uppermost part of the leg. After treatment the leg is easily and painlessly detached from the jig. Mice are observed on a daily basis between 11 and 30 days following treatment and the percentage of animals in each treatment group showing moist desquamation of the treated foot is recorded (Horsman et al., 1997). For lung irradiation, mice are under inhaled general anesthesia (isofluorane) when the left lung is irradiated. The right lung is shielded from radiation with lead and is used as a negative control. To assess lung damage, the pulmonary histology of mice that have survived for 8 months is studied after peptide associated radiation in the left lung. Both lungs are dissected and fixed in 10% formalin for 24 hours and sectioned into 5-μm-thick sections, mounted on glass slides, and stained. Masson's trichrome stain is used to detect fibrosis (Dileto and Travis, 1996).

ii. The Zebrafish Embryo Model

Zebrafish (Danio rerio) can be used as a unique vertebrate model to screen therapeutic agents rapidly and effectively because of their relatively close genetic relationship to humans, fecundity and accessibility, short embryonic development, and the ease of observation and direct visualization (Stern and Zon, 2003). Two zebrafish genes that are related to human genes have been cloned. Zebrafish ATM (zATM)(Garg et al., 2004) and zebrafish NBS1 (zNBS1, NCBI #AAW50708) share at least 70% of homology with human partners hATM and hNBS1. Several studies have investigated the developmental time and dose dependency of zebrafish embryo viability following exposure to ionizing radiation (Geiger et al., 2006; Traver et al., 2004; Berghmans et al., 2005). These studies have provided useful information for using this system to evaluate the radiosensitizing efficacy of the proposed NIP peptides.

Embryo harvesting and maintenance: Wild-type adult zebrafish are obtained and maintained according to standard operating procedures. Zebrafish are kept at 28.5° C. on a 14-hour day/10-hour night cycle. Adult fish are kept segregated by sex and mated in embryo collection tanks (Aquatic Habitats, Apopka, Fla.). Embryos from these breedings are collected soon after the onset of the light cycle and transferred to Petri dishes in 1 mM NaCl in tank water. Methylene blue is routinely added as antiseptic (0.5 mg/L final). Viable embryos are washed and sorted (10 embryos/well of standard 12-well culture plates) in 2 ml embryo medium by one- to two-cell stage (approximately 0.5-1 h post fertilization [hpf]), and maintained under normoxic conditions with temperature at 25° C. to slow normal development. EM is changed after dechorionation at 24-48 and again at 72-96 hpf.

Embryo irradiation and NGR-peptide exposure: Embryos at 2, 4, 6, 8, or 24 hpf are exposed to different doses (10-50 μM) of the NGR-peptides for one hour before they are exposed to single fractions of X-ray irradiation (5, 10 or 20 Gy). After irradiation, embryos are incubated at 25° C. for 24 h, dechorionated, and then maintained at 25° C. for up to 144 h to evaluate morphology and survival. It is noted here that both polyarginine conjugated peptides (R₉, wtNIP and scNIP) and NGR-conjugated peptides (NGR-only, NGR-wtNIP, and NGR-scNIP) are tested in the experiments.

Survival assays and morphological analysis: Survival of each embryo are continually assessed from the point of fertilization up to 144 hpf or the conclusion of each experiment. All observations are made using light microscopy. For the first 24 hpf, survival is determined through the assessment of appropriate cell division using the method described by Kimmel et al (Kimmel et al., 1995). After 24 hpf, cardiac contractility is defined as continued survival. Survival is calculated as a percentage of viable embryos to total number of embryos for each treatment group and survival curves represent the mean of three separate experiments. Radiosensitizing enhancement ratios is calculated as a survival ratio with the NIP-peptides pretreated embryos as the numerator and non-NIP-peptide pretreated embryos as the dominator. For histological evaluations, embryos are fixed in 4% paraformaldehyde and embedded in paraffin. Samples are sectioned and the tissue slices (5 μm) stained with H&E, assessed with a Leica microscope at ×40 magnification, and photographed using Q imaging Retiga Exi digital camera and analyzed with Image Pro Plus 5.1 software. At least 20 embryos from each treatment group are assessed, and the experiments repeated at least three times.

### 3. Example 3

### High Throughput Screening (HTS)

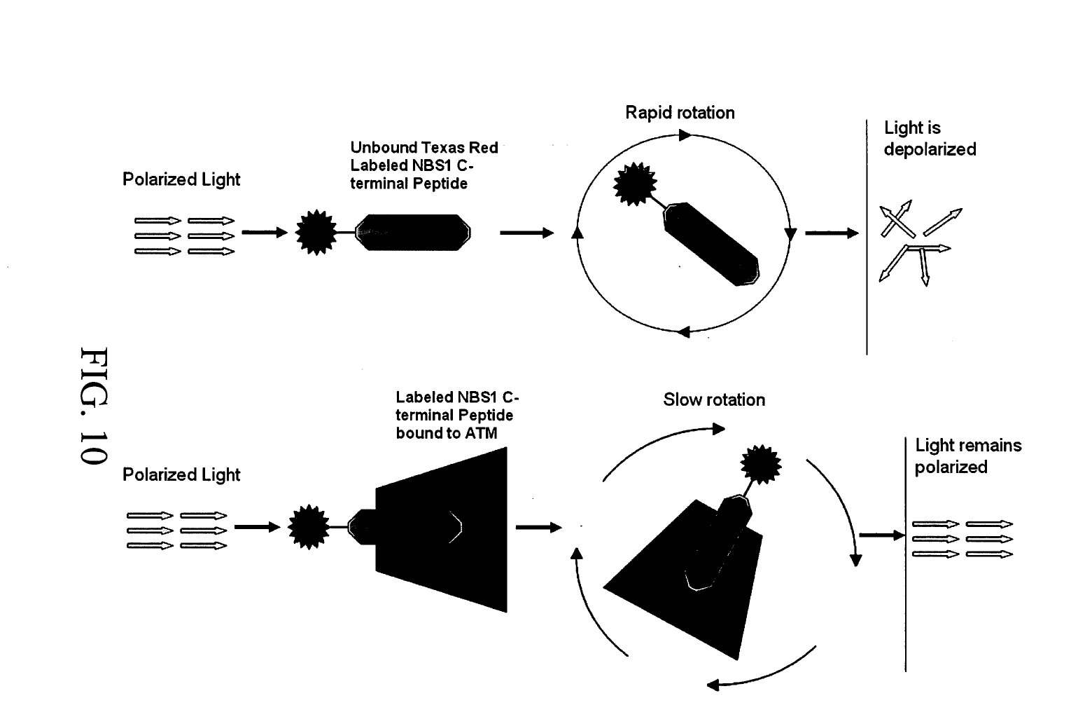

High throughput screening (HTS) has led to significant advances in the field of drug discovery, making it possible to screen huge libraries for chemical compounds that can disrupt protein-protein interactions and inhibit enzymatic activity (Fernandes, 1998; Sittampalam et al., 1997). One feasible approach for HTS assay development is Fluorescence Polarization (FP), which is a cell-free based assay for screening large molecular libraries (Roehrl et al., 2004). Fluorescence polarization (FP) assays make use of a fluorophore that is excited by polarized light, where only fluorophores that are parallel to the light are excited (Nasir and Jolley, 1999; Silverman et al., 1998). The rotational speed of the molecule is dependent on the size of the molecule, such that ligands less than 5000 Da can achieve significant depolarization, leading to rapid molecular rotation and emission of a depolarized fluorescent signal. For molecules of significantly larger size (>5000 Da), the ability of the fluorophore to depolarize light is severely reduced, resulting in an increase of the polarization signal (FIG. 10) (Sportsman et al., 2003; Thompson et al., 2002). Using FP HTS, inhibitors have been identified to target the BRCT domain of breast cancer related gene BRCA1, Hsp90, and Bcl-xL (Howes et al., 2006; Kim et al., 2004; Qian et al., 2004).

FP assay is used the to screen a library of compounds developed at Southern Research Institute (20K compounds) and the CB2 library (100K) and diversity sets from this library (10K and 3K), to identify compounds that can block NBS1-ATM interaction. Since it has been demonstrated that the conserved C-terminal of NBS1 binds to a series of the heat repeats (HR 2, a.a. 248-522, and HR 7, a.a. 1436-1770) in ATM (FIG. 1), the FP assay is achieved by detecting changes in florescence polarization signals in a cell-free assay with mixtures of GST-ATM peptides (as a receptor) and NBS1 peptides (as a tracer). A statistical experimental design is introduced for assay validation. Several parameters are validated and optimized, including receptor concentration, tracer concentration, plate type, DMSO tolerance, and incubation time.

i. Assay Development and Optimization

Generation and purification of GST-ATM peptides: A purified GST fusion protein containing HR 2 and HR 7 of ATM is generated. To produce the GST-ATM construct, an expression vector encoding a glutathione S-transferase (GST) containing residues 248-1770 (GST-ATM) is generated by inserting the corresponding PCR generated BamH1-EcoR1 fragment of human ATM cDNA into pGEX-2T (Amersham Bioscience). The GST-ATM fusion protein is purified by a standard GST-fusion peptide preparation method and protein homogeneity is analyzed by SDS-PAGE.

Effects of Texas Red labeling on NBS1-ATM binding: The second step is to determine an optimal position for Texas Red (TR) labeling on the NBS1 C-terminal peptide (a.a. 734-754). TR was chosen in place of fluorescein to eliminate false positives that may occur due to compound auto-fluorescence. First, whether TR labeling on the N-terminus or the C-terminus can affect NBS1-ATM binding is tested. The NBS1 conserved C-terminal sequence (QHAKEESLADDLFRYNPYLKRR, SEQ ID NO:3), which includes 3 critical binding sites for ATM (736-737 (EE), 741-742 (DD), and 745-746 (RY)), is used for the binding assay. To identify a site at which the TR labeling does not disrupt ATM binding, two peptides are synthesized with TR labels at either the N (TR-NBS1) or C-terminus (NBS1-TR) of the peptide as shown in Table 3. The dissociation constant, K_{d} is then determined for each labeled-peptide by titrating a constant concentration of TR-labeled peptide (100 μM) with increasing concentrations of the GST-ATM proteins. The GST tag is left in place since polarization is directly related to the molecular mass of the protein. The range of the assay is defined by the difference in polarization between the bound peptide and the free peptide, and the K_{d} value used to determine the best location for the TR label.

TABLE 3
Peptides used in FP-HTA assay development

| Peptide | Sequence | SEQ ID NO |
|---|---|---|
| TR-NBS1 | TR-QHAKEESLADDLFRYNPYLKRR | SEQ ID NO: 3 |
| NBS1-TR | QHAKEESLADDLFRYNPYLKRR-TR | SEQ ID NO: 3 |
| unlabeled | QHAKEESLADDLFRYNPYLKRR | SEQ ID NO: 3 |
| wtNBS1 | | |
| unlabeled | QHAKAASLAAALFAANPYLKRR | SEQ ID NO: 13 |
| mtNBS1 | | |

NBS1-ATM binding affinity, FP assay stability, and FP DMSO tolerance: To determine the ability of the labeled NBS1 peptide to bind to ATM, a competition based assay is conducted using the unlabeled wtNBS1 peptide of equal length to the labeled peptides (Table 1). The unlabeled NBS1 peptide is titrated into an optimum concentration (as defined previously) of GST-ATM and labeled NBS1 to determine the ability of the labeled NBS1 peptide to bind to ATM in the presence of unlabeled NBS1 peptide. The stability of the assay is an important parameter that determines the throughput of the FP screen. A time course study of NBS1-ATM binding is incorporated to determine the stability of the signal. The binding assay is incubated at room temperature over a 12 hour period with assay plate readings at 4 hour intervals over that time period. This study lends insight into the binding time of the NBS1-ATM complex. Since all of the chemical compounds in the screening libraries are dissolved in DMSO, testing the tolerance of DMSO (i.e., the concentration necessary to keep the compounds in solution without inhibiting the peptide-GST fusion protein interaction) is important in determining the final DMSO concentration in the FP assay for high throughput screening. DMSO with final concentrations ranging from 0-8% is added to each well before addition of the labeled NBS1 peptide and GST-ATM.

Biostatistical analysis of assay performance indicators: The Z′ factor evaluates the quality and suitability of the assay (0-1.0, 1.0 being the best assay, but 0.5-1.0 considered a robust assay) and is based on the mean (μ) and standard deviation (σ) of both positive (p) controls and negative (n) controls (μₚ, μₙ, σₚ, σₙ). To establish the Z′ factor, competitive inhibition using the FP assay will be used to establish the enzyme inhibitor constant Kᵢ. To establish the Kᵢ; values, increasing concentrations (0-500 μM) of unlabeled NBS1 peptide and 2 μM labeled NBS1 peptide are added to an appropriate concentration of GST-ATM. The concentration of GST-ATM is established from K_{d} values in the previous experiments which should be at least 1, and is based on the ratio between the receptor (ATM) and the K_{d} of the labeled NBS1 peptide. From these values, the Z′ factor is calculated according to:

$${Z}^{′} factor = 1 - \frac{3 \times ( {σ}_{p} - {σ}_{n} )}{\vert {μ}_{p} - {μ}_{n} \vert}$$

A Z′ factor that is ≧0.5 is considered acceptable for the FP high throughput screen.

The signal-to-noise ratio (S/N) is another important performance indication. S/N is used to quantify the extent of non-specific binding (NSB) where a signal to noise ratio of 10 or greater is an acceptable level of performance. However, in FP, the S/N cannot be used since the noise cannot be quantified by the extent of non-specific binding. In FP assays, unbound tracer is present and contributes to the overall NSB signal making it larger than the NSB signal alone. Therefore, the S/N value is smaller compared to other assays where unbound tracer must be removed or distinguished from bound tracer.

ii. HTS and Validation of Hits.

The diversity library established at Southern Research Institute (20,000 compounds) and the Chembridge library (100,000 compounds+30,000 and 20,000 diversity sets within this library) are screened at 10 μM with a 10 μμM TR-labeled NBS1 peptide and an optimal concentration (as defined in previous experiments) of GST-ATM in DMSO solution in 15 minutes incubation at room temperature. The assay is carried out in 384 or 1536-well plates with the unlabeled wtNIP peptide used as a positive inhibitory control. The hits identified from this experiment are serially diluted, and the assay repeated and the Kᵢ values established. The compounds with the 10 lowest Kᵢ value are chosen for further evaluation.

iii. In vitro Evaluation of the Hit Compounds.

Once compounds that can best inhibit NBS1-ATM interaction are identified, these compounds are tested in tissue culture models. First, those compounds that are already known to be cytotoxic and/or radiosensitizers are identified. These known compounds are ruled out for further in vitro studies. The remaining compounds are first assessed for cytotoxicity using the MTT assay and the colony formation in several tumor cell lines, including Hela, MCF-7 and PC-3. Those compounds that do not possess cytotoxicity for radiosensitizing studies are chosen. The radiosensitization assay is performed using colony formation assay to assess radiation induced survival. IR-induced γH2AX and NBS1 foci formation is assessed in the presence of the compounds.

### 4. Example 4

### Gene Delivery of Radiosensitizing Peptide

Hypoxic tumor cells are considered to be hyper-resistant to radiation, leading to a failure of radiotherapy. If the wtNIP radiosensitizing peptide can be expressed specifically in hypoxic tumor tissues, then a dramatic increase of tumor control by radiotherapy is expected. To reach this goal, a hypoxia-driven adenovirus vector is provided that can express wtNIP in hypoxic tissues. The efficacy of the virus in both tissue culture and animal models is assessed.

First, an expression cassette in which the hypoxia-response promoter drives wtNIP expression is generated. A complement pair of two synthetic oligonucleotides are generated based on the hypoxia response enhancer element from the murine phosphoglycerate kinase I 5′ flanking sequence (−307 to −290) with NheI compatible ends:

(SEQ ID NO: 14)

5′CTAGAGTCGTGCAGGACGTGACATCTAGTGTCGTGCAGGCATCTAGTG
TCGTGCAGGACGTGCATC3′,

(SEQ ID NO: 15)

3′TCAGCACGTCCTGCACTGTAGATCACAGCACGTCCGTAGATCACAGCA
CGTCCTGCACTGTAGGATC5′.

The two oligos are annealed to form a double strand DNA and cloned directly to the NheI site of the pGL3-promoter vector (Promega, Madison, Wis.). The pGL3-promoter vector has an SV40 basic promoter, and the structure of the hypoxia response enhancer in combination with the SV40 basic promoter was proven to drive reporter genes and tumor suicide genes to specifically express in hypoxic tumors. Then, another pair of oligos are made based on the small peptide sequence with 5′ Nco 1 and 3′ Xbal compatible sites. For easy detection of the small peptide expression, a His tag sequence linked to 3′ end of the peptide is designed. The oligo sequences are:

(SEQ ID NO: 16)

5′CATGGAAGGAGGAAGCAGCCAAGGAGAAG/ACCACCACCACCACCACC
AC/-3′,

(SEQ ID NO: 17)

3′CTTCCTCCTTCGTCGGTTCCTCTTCT/GGTGGTGGTGGTGGTGGTG/G
ATC-5′.

The sequence between the slashes is a tandem repeat of 6 histidines. After being annealed to a double strand DNA, the fragment is directly cloned into the Nco I and Xbal sites of the pGL-3 promoter vector to replace the original luciferase reporter gene. The resultant construct is confirmed by sequence. The whole expression cassette, released by Kpn I and BamH I, is ligated into the adenovirus vector shuttle plasmid (Stratagene, Calif.). Then, the shuttle vector is recombined by homologous recombination with the E1- and E3-deleted pAdEasy-1 adenoviral backbone vector to generate a packagable Ad genome. To achieve efficient recombination, BJ5183 competent bacteria is transformed by electroporation, and the correct clone plasmid, pAd5-hypoxia-SIP2, is chosen for adenovirus vector production. To generate the adenovector, the 911 adenovirus packaging cell line is used and transfected by calcium phosphate precipitation. The produced vector is propagated in 911 cells. Cesium chloride gradient centrifugation and Sepharose CL-6B colume desalting is performed to concentrate and purify the vector preparation.

After the vector is generated, it is expressed in hypoxic human cancer cell cultures to test whether they express the NIPs. After expression is confirmed, a radiosensitization effect is investigated. In addition, the viruses are injected in the mouse xenograft model and the in vivo radiosensitizing effect evaluated.

### B. REFERENCES

Abraham, R. T. (2001). Cell cycle checkpoint signaling through the ATM and ATR kinases. Genes & Development 15, 2177-2196.
Adam, E. and Nasz, I. (2001). [Adenovirus vectors and their clinical application in gene therapy]. Orv. Hetil. 142, 2061-2070.
Arap, W., Haedicke, W., Bernasconi, M., Kain, R., Rajotte, D., Krajewski, S., Ellerby, H. M., Bredesen, D. E., Pasqualini, R., and Ruoslahti, E. (2002). Targeting the prostate for destruction through a vascular address. Proc. Natl. Acad. Sci. U.S. A 99, 1527-1531.
Arap, W., Pasqualini, R., and Ruoslahti, E. (1998). Cancer Treatment by Targeted Drug Delivery to Tumor Vasculature in a Mouce Model. Science 279, 377-380.
Bakkenist, C. J. and Kastan, M. B. (2003). DNA damage activates ATM through intermolecular autophosphorylation and dimer dissociation. Nature 421, 499-506.
Berghmans, S., Murphey, R. D., Wienholds, E., Neuberg, D., Kutok, J. L., Fletcher, C. D., Morris, J. P., Liu, T. X., Schulte-Merker, S., Kanki, J. P., Plasterk, R., Zon, L. I., and Look, A. T. (2005). tp53 mutant zebrafish develop malignant peripheral nerve sheath tumors. Proc. Natl. Acad. Sci. U.S. A 102, 407-412.
Burma, S., Chen, B. P., Murphy, M., Kurimasa, A., and Chen, D. J. (2001). ATM phosphorylates histone H2AX in response to DNA double-strand breaks. J. Biol. Chem. 276, 42462-42467.
Cerosaletti, K. and Concannon, P. (2004). Independent roles for nibrin and Mre11-Rad50 in the activation and function of Atm. J. Biol. Chem. 279, 38813-38819.
Cerosaletti, K., Wright, J., and Concannon, P. (2006). Active role for nibrin in the kinetics of atm activation. Mol. Cell. Biol. 26, 1691-1699.
Cerosaletti, K. M. and Concannon, P. (2003). Nibrin forkhead-associated domain and breast cancer C-terminal domain are both required for nuclear focus formation and phosphorylation. J. Biol. Chem. 278, 21944-21951.
Choudhury, A., Cuddihy, A., and Bristow, R. G. (2006). Radiation and new molecular agents part I: targeting ATM-ATR checkpoints, DNA repair, and the proteasome. Semin. Radiat. Oncol. 16, 51-58.
Curnis, F., Arrigoni, G., Sacchi, A., Fischetti, L., Arap, W., Pasqualini, R., and Corti, A. (2002). Differential binding of drugs containing the NGR motif to CD13 isoforms in tumor vessels, epithelia, and myeloid cells. Cancer Res. 62, 867-874.
Derossi, D., Calvet, S., Trembleau, A., Brunissen, A., Chassaing, G., and Prochiantz, A. (1996). Cell internalization of the third helix of the Antennapedia homeodomain is receptor-independent. J. Biol. Chem. 271, 18188-18193.
Deshayes, S., Morris, M. C., Divita, G., and Heitz, F. (2005). Cell-penetrating peptides: tools for intracellular delivery of therapeutics. Cell Mol. Life. Sci. 62, 1839-1849.
Difilippantonio, S., Celeste, A., Fernandez-Capetillo, O., Chen, H. T., Reina, S. M., Van Laethem, F., Yang, Y. P., Petukhova, G. V., Eckhaus, M., Feigenbaum, L., Manova, K., Kruhlak, M., Camerini-Otero, R. D., Sharan, S., Nussenzweig, M., and Nussenzweig, A. (2005). Role of Nbs1 in the activation of the Atm kinase revealed in humanized mouse models. Nat. Cell Biol. 7, 675-685.
Dileto, C. L. and Travis, E. L. (1996). Fibroblast radiosensitivity in vitro and lung fibrosis in vivo: comparison between a fibrosis-prone and fibrosis-resistant mouse strain. Radiat. Res. 146, 61-67.
Dornan, D., Shimizu, H., Mah, A., Dudhela, T., Eby, M., O'Rourke, K., Seshagiri, S., and Dixit, V. M. (2006). ATM engages autodegradation of the E3 ubiquitin ligase COP1 after DNA damage. Science 313, 1122-1126.
Dupre, A., Boyer-Chatenet, L., and Gautier, J. (2006). Two-step activation of ATM by DNA and the Mre11-Rad50-Nbs1 complex. Nat. Struct. Mol. Biol. 13, 451-457.
Ellerby, H. M., Arap, W., Ellerby, L. M., Kain, R., Andrusiak, R., Del Rio, G., Krajewski, S., Lombardo, C. R., Rao, R., Ruoslahti, E., Bredesen, D. E., and Pasqualini, R. (1999). Anti-cancer activity of targeted pro-apoptotic peptides. Nature Medicine 5, 1032-1038.
Falck, J., Coates, J., and Jackson, S. P. (2005). Conserved modes of recruitment of ATM, ATR and DNA-PKcs to sites of DNA damage. Nature 434, 605-611.
Fan, Z., Chakravarty, P., Alfieri, A., Pandita, T. K., Vikram, B., and Guha, C. (2000). Adenovirus-mediated antisense ATM gene transfer sensitizes prostate cancer cells to radiation. Cancer Gene Ther. 7, 1307-1314.
Fernandes, P. B. (1998). Technological advances in high-throughput screening. Curr. Opin. Chem. Biol. 2, 597-603.
Fuchs, S. M. and Raines, R. T. (2004). Pathway for polyarginine entry into mammalian cells. Biochemistry 43, 2438-2444.
Furuta, T., Takemura, H., Liao, Z. Y., Aune, G. J., Redon, C., Sedelnikova, O. A., Pilch, D. R., Rogakou, E. P., Celeste, A., Chen, H. T., Nussenzweig, A., Aladjem, M. I., Bonner, W. M., and Pommier, Y. (2003). Phosphorylation of histone H2AX and activation of Mre11, Rad50, and Nbs1 in response to replication-dependent DNA double-strand breaks induced by mammalian DNA topoisomerase I cleavage complexes. J. Biol. Chem. 278, 20303-20312.
Garg, R., Geng, C. D., Miller, J. L., Callens, S., Tang, X., Appel, B., and Xu, B. (2004). Molecular cloning and characterization of the catalytic domain of zebrafish homologue of the ataxia-telangiectasia mutated gene. Mol Cancer Res. 2, 348-353.
Geiger, G. A., Parker, S. E., Beothy, A. P., Tucker, J. A., Mullins, M. C., and Kao, G. D. (2006). Zebrafish as a ‘biosensor’? Effects of ionizing radiation and amifostine on embryonic viability and development. Cancer Res. 66, 8172-8181.
Glasgow, J. N., Everts, M., and Curiel, D. T. (2006). Transductional targeting of adenovirus vectors for gene therapy. Cancer Gene Ther. 13, 830-844.
Guha, C., Guha, U., Tribius, S., Alfieri, A., Casper, D., Chakravarty, P., Mellado, W., Pandita, T. K., and Vikram, B. (2000). Antisense ATM gene therapy: a strategy to increase the radiosensitivity of human tumors. Gene Therapy 7, 852-858.
Hickson, I., Zhao, Y., Richardson, C. J., Green, S. J., Martin, N. M., Orr, A. I., Reaper, P. M., Jackson, S. P., Curtin, N. J., and Smith, G. C. (2004). Identification and characterization of a novel and specific inhibitor of the ataxia-telangiectasia mutated kinase ATM. Cancer Res. 64, 9152-9159.
Horsman, M. R., Siemann, D. W., Chaplin, D. J., and Overgaard, J. (1997). Nicotinamide as a radiosensitizer in tumours and normal tissues: the importance of drug dose and timing. Radiother. Oncol. 45, 167-174.
Howes, R., Barril, X., Dymock, B. W., Grant, K., Northfield, C. J., Robertson, A. G., Surgenor, A., Wayne, J., Wright, L., James, K., Matthews, T., Cheung, K. M., McDonald, E., Workman, P., and Drysdale, M. J. (2006). A fluorescence polarization assay for inhibitors of Hsp90. Anal. Biochem. 350, 202-213.
Jones, S. W., Christison, R., Bundell, K., Voyce, C. J., Brockbank, S. M., Newham, P., and Lindsay, M. A. (2005). Characterisation of cell-penetrating peptide-mediated peptide delivery. Br. J. Pharmacol. 145, 1093-1102.
Kim, J., Felts, S., Llauger, L., He, H., Huezo, H., Rosen, N., and Chiosis, G. (2004). Development of a fluorescence polarization assay for the molecular chaperone Hsp90. J. Biomol. Screen. 9, 375-381.
Kimmel, C. B., Ballard, W. W., Kimmel, S. R., Ullmann, B., and Schilling, T. F. (1995). Stages of embryonic development of the zebrafish. Dev. Dyn. 203, 253-310.
Lee, J. H. and Paull, T. T. (2004). Direct activation of the ATM protein kinase by the Mre11/Rad50/Nbs1 complex. Science 304, 93-96.
Lee, J. H. and Paull, T. T. (2005). ATM activation by DNA double-strand breaks through the Mre11-Rad50-Nbs1 complex. Science 308, 551-554.
Lim, D.-S., Kim, S.-T., Xu, B., Maser, R. S., Lin, J., Petrini, J. H. J., and Kastan, M. B. (2000). ATM phosphorylates p95/nbs1 in an S-phase checkpoint pathway. Nature 404, 613-617.
McManus, K. J. and Hendzel, M. J. (2005). ATM-dependent DNA damage-independent mitotic phosphorylation of H2AX in normally growing mammalian cells. Mol. Biol. Cell 16, 5013-5025.
Nasir, M. S, and Jolley, M. E. (1999). Fluorescence polarization: an analytical tool for immunoassay and drug discovery. Comb. Chem. High Throughput. Screen. 2, 177-190.
Pasqualini, R., Koivunen, E., Kain, R., Landenranta, J., Sakamoto, M., Stryhn, A., Ashmun, R. A., Shapiro, L. H., Arap, W., and Ruoslahti, E. (2000). Aminopeptidase N is a receptor for tumor-homing peptides and a target for inhibiting angiogenesis. Cancer Res. 60, 722-727.
Pasqualini, R., Koivunen, E., and Ruoslahti, E. (1997). Alpha v integrins as receptors for tumor targeting by circulating ligands. Nat. Biotechnol. 15, 542-546.
Pawlik, T. M. and Keyomarsi, K. (2004). Role of cell cycle in mediating sensitivity to radiotherapy. Int. J. Radiat. Oncol. Biol. Phys. 59, 928-942.
Pellegrini, M., Celeste, A., Difilippantonio, S., Guo, R., Wang, W., Feigenbaum, L., and Nussenzweig, A. (2006). Autophosphorylation at serine 1987 is dispensable for murine Atm activation in vivo. Nature 443, 222-225.
Qian, J., Voorbach, M. J., Huth, J. R., Coen, M. L., Zhang, H., Ng, S. C., Comess, K. M., Petros, A. M., Rosenberg, S. H., Warrior, U., and Burns, D. J. (2004). Discovery of novel inhibitors of Bcl-xL using multiple high-throughput screening platforms. Anal. Biochem. 328, 131-138.
Roehrl, M. H., Wang, J. Y., and Wagner, G. (2004). A general framework for development and data analysis of competitive high-throughput screens for small-molecule inhibitors of protein-protein interactions by fluorescence polarization. Biochemistry 43, 16056-16066.
Ruoslahti, E. (2000). Targeting tumor vasculature with homing peptides from phage display. Semin Cancer Biol. 10, 435-442.
Savitsky, K., Bar-Shira, A., Gilad, S., Rotman, G., Ziv, Y., Vanagaite, L., Tagle, D. A., Smith, S., Uziel, T., Sfez, S., Ashkenazi, M., Pecker, I., Frydman, M., Harnik, R., Patanjali, S. R., Simmons, A., Clines, G. A., Sartiel, A., Gatti, R. A., Chessa, L., Sanal, O., Lavin, M. F., Jaspers, N. G. J., Taylor, A. M. R., Arlett, C. F., Miki, T., Weissman, S. M., Lovett, M., Collins, F. S., and Shiloh, Y. (1995). A Single Ataxia Telangiectasia Gene with A Product Similar to PI-3 Kinase. Science 268, 1749-1753.
Schwarze, S. R., Ho, A., Vocero-Akbani, A., and Dowdy, S. F. (1999). In vivo protein transduction: Delivery of a biologically active protein into the mouse. Science 285, 1569-1572.
Shiloh, Y. (2003). ATM and related protein kinases: safeguarding genome integrity. Nat Rev Cancer 3, 155-168.
Silverman, L., Campbell, R., and Broach, J. R. (1998). New assay technologies for high-throughput screening. Curr. Opin. Chem. Biol. 2, 397-403.
Sittampalam, G. S., Kahl, S. D., and Janzen, W. P. (1997). High-throughput screening: advances in assay technologies. Curr. Opin. Chem. Biol. 1, 384-391.
Sportsman, J. R., Daijo, J., and Gaudet, E. A. (2003). Fluorescence polarization assays in signal transduction discovery. Comb. Chem. High Throughput. Screen. 6, 195-200.
Stern, H. M. and Zon, L. I. (2003). Cancer genetics and drug discovery in the zebrafish. Nat Rev Cancer 3, 533-539.
Takenobu, T., Tomizawa, K., Matsushita, M., Li, S. T., Moriwaki, A., Lu, Y. F., and Matsui, H. (2002). Development of p53 protein transduction therapy using membrane-permeable peptides and the application to oral cancer cells. Mol. Cancer. Ther. 1, 1043-1049.
Thompson, R. B., Gryczynski, I., and Malicka, J. (2002). Fluorescence polarization standards for high-throughput screening and imaging. BioTechniques 32, 34, 37-8, 40, 42.
Traver, D., Winzeler, A., Stern, H. M., Mayhall, E. A., Langenau, D. M., Kutok, J. L., Look, A. T., and Zon, L. I. (2004). Effects of lethal irradiation in zebrafish and rescue by hematopoietic cell transplantation. Blood 104, 1298-1305.
Yuan, J. P., Kramer, A., Eckerdt, F., Kaufmann, M., and Strebhardt, K. (2002). Efficient internalization of the polo-box of polo-like kinase 1 fused to an antennapedia peptide results in inhibition of cancer cell proliferation. Cancer Research 62, 4186-4190.
Zhang, N., Chen, P., Gatei, M., Scott, S., Khanna, K. K., and Lavin, M. F. (1998). An anti-sense contruct of full-length ATM cDNA imposes a radiosensitive phenotype on normal cells. Oncogene 17, 811-818.

What is claimed is:

1. An isolated peptide comprising the carboxy-terminal amino acid sequence of NBS1, or a conservative variant thereof, wherein the polypeptide does not comprise the full length NBS1.

2. The polypeptide of claim 1, wherein polypeptide comprises an amino acid sequence with at least 95% sequence identity to SEQ ID NO:1.

3. The polypeptide of claim 1 or 2, wherein the polypeptide inhibits the binding of ATM to the carboxy-terminus of NBS1.

4. The polypeptide of any of claims 1 to 3, wherein the polypeptide comprises from 4 to 30 contiguous amino acids of the carboxy-terminus of NBS1.

5. The polypeptide of claim 1, wherein the polypeptide comprises amino acids 734 to 744 of NBS1 (SEQ ID NO:1).

6. The polypeptide of any of claims 1 to 5, wherein the polypeptide comprises a conservative amino acid substitution within amino acids 734 to 754 of NBS1.

7. The polypeptide of any of claims 1 to 6, wherein the polypeptide comprises an amino acid sequence with at least 95% sequence identity to SEQ ID NO:3.

8. The polypeptide of any of claims 1 to 7, wherein the polypeptide comprises the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

9. The polypeptide of any of claims 1 to 8, further comprising a cellular internalization sequence.

10. The polypeptide of claim 9, wherein the cellular internalization comprises an amino acid sequence of a protein selected from a group consisting of Polyarginine, Antennapedia, TAT, HIV-Tat, Penetratin, Antp-3A (Antp mutant), Buforin Transportan, MAP (model amphipathic peptide), K-FGF, Ku70, Prion, pVEC, Pep-1, SynB1, Pep-7, HN-1, BGSC (Bis-Guanidinium-Spermidine-Cholesterol and BGTC (Bis-Guanidinium-Tren-Cholesterol.

11. The polypeptide of any of claims 1 to 10, wherein the polypeptide comprises the amino acid sequence selected from the group consisting of SEQ ID NO:35 SEQ ID NO:36, SEQ ID NO:37, SEQ ID NO:38, SEQ ID NO:39, SEQ ID NO:40, SEQ ID NO:41, and SEQ ID NO:42.

12. The polypeptide of any of claims 1 to 11, further comprising a tumor specific targeting sequence.

13. The polypeptide of claim 12, wherein the tumor specific targeting sequence comprises an RGD, NGR, or GSL motif.

14. The polypeptide of claim 13, wherein polypeptide comprises the amino acid sequence set forth in SEQ ID NO:11 or SEQ ID NO:12.

15. An isolated nucleic acid encoding the polypeptide of claim 1.

16. The isolated nucleic acid of claim 15, wherein the encoded polypeptide comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

17. The isolated nucleic acid of claim 16, comprising the nucleic acid sequence SEQ ID NO:43, SEQ ID NO:44, SEQ ID NO:45, SEQ ID NO:46, SEQ ID NO:47, SEQ ID NO:48, SEQ ID NO:49, and SEQ ID NO:50.

18. The isolated nucleic acid of one any of claims 15 to 17, wherein the nucleic acid is operably linked to an expression control sequence.

19. A vector comprising the nucleic acid of any of claims 15 to 17 operably linked to an expression control sequence.

20. The vector of claim 19, wherein the vector is a viral vector.

21. The vector of claim 20, wherein the vector is an adenovirus vector.

22. A cell comprising the nucleic acid of any one of claims 15 to 17.

23. A cell comprising the vector of claim 19.

24. An organism comprising the nucleic acid of one claims 15 to 17.

25. An organism comprising the vector of claim 19.

26. A composition comprising the polypeptide of one claims 1 to 14 in a pharmaceutically acceptable carrier.

27. A composition comprising the nucleic acid of one claims 15 to 17 in a pharmaceutically acceptable carrier.

28. A composition comprising the vector of one claims 19 to 21 in a pharmaceutically acceptable carrier.

29. A method of increasing the sensitivity of a tissue to radiotherapy, the steps of the method comprising:
a) administering to the tissue a composition that inhibits the interaction of NBS1 with ATM, and
b) irradiating the tissue.

30. The method of claim 29, wherein the tissue comprises a benign growth.

31. The method of claim 29, wherein the tissue comprises a cancer.

32. A method of treating cancer in a subject, the steps of the method comprising:
a) administering to the cancer a composition that inhibits the interaction of NBS1 with ATM, and
b) irradiating the cancer.

33. A method of identifying a radiosensitizing agent, the steps of the method comprising:
a) contacting a sample comprising NBS1 and ATM polypeptides with a candidate agent, and
b) detecting the interaction between the NBS1 and ATM polypeptides, a decrease in the interaction between the NBS1 and ATM polypeptides as compared to controls indicating the candidate agent is radiosensitizing.

34. The method of claim 33, wherein the interaction between the NBS1 and ATM polypeptides is detected using fluorescence polarization.

35. The method of claim 34, wherein the NBS1 or ATM polypeptide comprises a fluorophore

36. The method of any one of claims 33 to 35, wherein the polypeptide of one claims 1 to 14 is used as a positive control.

37. A method of treating cancer in a subject, the steps of the method comprising:
a) administering to the cancer a composition that inhibits the interaction of NBS1 with ATM, and
b) administering to the cancer an anti-neoplastic drug.
